(19) United States
(12) Patent Application Publication
(10) Pub. No.: US 2022/0072198 A1
(43) Pub. Date: Mar. 10, 2022

(54) Titanium Dioxide Coatings for Medical Devices Made by Atomic Layer Deposition

(71) Applicant: Northeastern University, Boston, MA (US)

(72) Inventors: Thomas Jay WEBSTER, Barrington, RI (US); Paria GHANNADIAN, Cambridge, MA (US); Fan YANG, Pittsburgh, PA (US); James Walter MOXLEY, Cambridge, MA (US)

(21) Appl. No.: 17/421,618

(22) PCT Filed: Jan. 10, 2020
(86) PCT No.: PCT/US2020/013238

§ 371 Date: Jul. 8, 2021

Related U.S. Application Data
(60) Provisional application No. 62/790,999, filed on Jan. 10, 2019.

(51) Int. Cl. A61L 27/30; C23C 16/02; C23C 16/40; A61L 27/06; A61L 27/04; C23C 16/455; C23C 16/52; A61F 2/82

(52) U.S. Cl. CPC A61L 27/306; C23C 16/02; C23C 16/405; A61L 27/06; A61L 27/047; A61L 2400/18; C23C 16/52; A61F 2/82; A61L 2420/08; A61L 2420/02; C23C 16/45527

(57) ABSTRACT

Implantable medical devices coated with multiple atomic layers of amorphous titanium dioxide applied by atomic layer deposition have improved mammalian cell adhesion and inhibition of bacterial growth. Thickness of the coating can be used to tune resorption of bioresorbable vascular scaffolds for treatments of cardiovascular disease.

### CROSS-REFERENCE TO RELATED APPLICATIONS

This application claims priority to U.S. Provisional Application No. 62/790,999, filed 10 Jan. 2019, the entirety of which is incorporated herein by reference.

### BACKGROUND

Coronary arteries can be blocked or narrowed by a buildup of plaque which results in the reduction of blood flow to the heart and causes chest discomfort. In some cases, blood clots can suddenly form inside the coronary arteries to cause a complete block of blood flow which leads to a heart attack. If coronary artery narrowing occurs, a stent may be required to reopen a blocked artery. Coronary stents are widely used in coronary artery disease (CAD) or coronary heart disease (CHD) treatments, keeping arteries open to support blood supply. The surgical procedure to insert a coronary stent, percutaneous coronary intervention (PCI), requires a guideline to lead a coronary stent to plaque on the artery inner wall. After placement, the stent expands to compress the plaque and restore normal blood flow inside the artery.

Coronary stents are now used in more than 90% of PCI procedures [1] and have evolved from balloon angioplasty to bare metal stents, drug-eluting stents, and recently to bioresorbable vascular scaffolds. Balloon angioplasty did not initially involve stent deployment [2]. Because of re-narrowing of coronary arteries due to acute vessel closure, bare metal stents were created to temporarily support narrowed arteries. The first Food and Drug Administration approved balloon-expandable slotted tube device, Palmaz-Schatz®, was invented by Johnson & Johnson [3]. The bare metal device was made of stainless steel and remained one of the most studied and widely used stents in the 1990s. However, the metallic density was high, which resulted in a high risk of sub-acute stent thrombosis. The technical challenges to implant bare metal stents also resulted in frequent surgery failures of stent placement and embolization [4]. After upgrades in both surgical and stent device technologies, drug eluting stents brought a new revolution to interventional cardiology. A drug eluting stent is a metal stent having a coating that elutes an anti-proliferative drug such as sirolimus, paclitaxel, or everolimus, which can substantially reduce the rate of in-stent restenosis compared with bare metal stents [5].

Currently, permanent metal and polymer scaffolds are implanted into coronary arteries to function as a long-term (>1 year) vascular stents. However, chronic or long-term clinical issues may occur due to the toxicity of implant materials, since these materials cannot be safely absorbed by the human body. For example, contemporary metallic drug-eluting stents have good clinical outcomes within 1 year of implantation. After 1 year, stent-related adverse events may appear, such as thrombosis, restenosis, and even myocardial infarction. Additionally, chronic inflammation, neoatherosclerosis, and strut fracture may affect the whole human body. Further surgery may be required to remove the stent, introducing risk for plaque buildup and requiring more stents to be placed in the artery [5].

The bioresorbable vascular scaffold is an alternative solution specially designed for stent implantation as the scaffold can be fully absorbed by the body safely, thereby eliminating the need of secondary surgeries to remove permanent stents and the associated risk of further chronic diseases. The complete life cycle of bioresorbable vascular scaffolds includes three phases: revascularization, restoration, and resorption. Revascularization involves alleviating coronary stenosis ischemia and is similar to drug eluting stents in which drug elution occurs within the first 5-6 months. Restoration refers to when the scaffold starts to experience mass loss followed by a reduction in molecular weight after 6 months of implantation. Finally, depending on the degradation rate of the stent, the resorption process can take up to 2-4 years. Recovery of vascular structure and function occurs within the revascularization process. After the scaffold has remodeled the coronary artery, it starts to disappear throughout the next two phases of the BVS life cycle. The FDA has approved only one bioresorbable vascular scaffold [6], which uses poly(lactic acid) (PLLA) as the stent platform. This scaffold has been reported to show positive vessel remodeling and plaque regression during the resorption process between 1 and 5 years after implantation [7, 8]. However, polymeric stents in general have a lower tensile strength, reduced stiffness, and reduced ductility compared to metallic stents. Also, polymeric drug eluting stents have been reported to have late thrombosis clinical issues [5]. On the other hand, metallic biomaterials are very popular for biomedical applications research.

Magnesium alloys have desirable mechanical properties and biocompatibility. Magnesium ions present in these alloys participate in many metabolic reactions and biological mechanisms. The large amount of magnesium present in the human body lends biocompatibility to Mg alloys. Normally, the human body contains approximately 35 g of Mg per 70 kg of body weight and the daily intake of Mg is about 375 mg [9]. A key feature of Mg for biomedical applications is that it is biodegradable. Magnesium alloys have advantages over traditional ceramics, biodegradable polymers, and other metallic materials. With magnesium's excellent mechanical properties of light weight, high mechanical strength, and high fracture toughness, many types of Mg stents have been used since 2004.

Biotronik introduced three generations of absorbable metal stents with WE43 magnesium alloy as the platform. The first clinical study involving 63 patients reported these to have safely degraded after four months. The third generation of AMS was coated with a degradable polymer carrier with antiproliferative drug and showed positive results of safety and efficacy compared to previous absorbable metal stents during in vivo trials [10]. However, WE43 contains 4% Yttrium and 2.25%, rare earth metals, which can be toxic to the human body.

The possibility to coat less toxic materials exists and can provide better outcomes for patients. Thus, there is a need to develop new biomaterials and coatings that are either non-toxic or have low toxicity for producing new generations of bioresorbable vascular scaffolds.

### SUMMARY

The present technology provides a process for chemically depositing a TiO₂ coating of nanoscale thickness on a variety of substrates including metals and metal alloys, such as those found on surfaces of implantable medical devices. The technology can be used to apply TiO₂ nanoscale films to biocompatible and bioresobable alloys, such as magnesium-zinc (Mg—Zn) alloy used in bioresorbable vascular scaffolds (BVS). The coatings provided by the technology endow surfaces of implanted medical devices with improved adsorption of cells of the subject while inhibiting the growth of bacteria and promoting wound healing and integration of an implanted device.

An aspect of the technology is an implantable medical device coated at least in part with a titanium dioxide coating that contains two or more single atomic layers of titanium dioxide. The coating is deposited by atomic layer deposition and provides 2 or more, 10 or more, 100 or more, 500 or more, 1000 or more, 2000 or more, 3000 or more, or 5000 or more individual atomic layers of titanium dioxide, each having a thickness of about 0.4 angstroms. The coating can contain amorphous titanium dioxide. The device can be, for example, a stent, stimulator, catheter, pacemaker, defibrillator, lead, electrode, bone fixation device, screw, pin, orthopedic implant, dental implant, pump, or prosthesis.

Another aspect of the technology is a method of treating a medical condition in a subject that includes implanting the implantable medical device described above into the subject's body. The medical condition can be, for example, coronary artery disease, cardiac arrhythmia, a spinal condition, broken bone, torn ligament, a dental condition, urinary obstruction, a prostate condition, cancer, diabetes, or chronic pain. When implanted, the titanium dioxide coating of the device can promote the adhesion, growth, and proliferation of cells of the patient on or near the device, and/or can inhibit the attachment, growth, and proliferation of bacteria or the growth of a bacteria-laden biofilm on the device.

The present technology can be further summarized in the following list of features.

1. An implantable medical device coated at least in part with a titanium dioxide coating, wherein the coating comprises two or more single atomic layers of titanium dioxide.
2. The implantable medical device of feature 1, wherein the titanium dioxide coating comprises amorphous titanium dioxide.
3. The implantable medical device of feature 1 or 2, wherein each of said single atomic layers has a thickness of about 0.4 angstroms.
4. The implantable medical device of any of the previous features, wherein the coating comprises about 600 to about 3250 single atomic layers of titanium dioxide.
5. The implantable medical device of any of the previous features, wherein the thickness of the titanium dioxide coating is in the range from about 70 nm to about 130 nm.
6. The implantable medical device of feature 5, wherein the coating comprises about 2500 single atomic layers of titanium dioxide and has a thickness of about 100 nm.
7. The implantable medical device of any of the previous features, wherein the titanium dioxide coating has an rms surface roughness from about 25 nm to about 65 nm, or from about 30 nm to about 45 nm.
8. The implantable medical device of any of the previous features, wherein the device comprises a metal or metal alloy coated at least in part with said titanium dioxide coating.
9 The implantable medical device of feature 8, wherein the metal or metal alloy is selected from the group consisting of Mg—Zn, Ti—V—Al, Ti, and Mg.
10. The implantable medical device of any of the previous features, wherein the device comprises a bioresorbable material coated at least in part with said titanium dioxide coating.
11. The implantable medical device of feature 10, wherein the device is a bioresorbable vascular scaffold.
12. The implantable medical device of any of the previous features, wherein the implantable medical device is selected from the group consisting of a stent, stimulator, catheter, pacemaker, defibrillator, lead, electrode, bone fixation device, screw, pin, orthopedic implant, dental implant, pump, or prosthesis.
13. The implantable medical device of feature 12, wherein the device is a vascular stent, and wherein the titanium dioxide coating is operative to extend the restoration time and/or the resorption time resulting from the stent when implanted in a vessel.
14. The implantable vascular device of feature 13, wherein the extension of the restoration time and/or the resorption time is modulated by the thickness of the titanium dioxide coating.
15. The implantable medical device of any of the previous features, wherein the titanium dioxide coating promotes adhesion of mammalian cells to the titanium dioxide coating.
16. The implantable medical device of any of the previous features, wherein the titanium dioxide coating promotes proliferation of mammalian cells on the titanium dioxide coating.
17. The implantable medical device of any of the previous features, wherein the titanium dioxide coating inhibits growth of bacteria on the titanium dioxide coating.
18. The implantable medical device of any of the previous features, wherein the titanium dioxide coating is deposited using two or more cycles of atomic layer deposition (ALD).
19. A method of treating a medical condition in a subject, the method comprising implanting the implantable medical device of any of features 1-18 into the subject's body.
20. The method of feature 19, wherein the medical condition is selected from the group consisting of coronary artery disease, cardiac arrhythmia, a spinal condition, broken bone, torn ligament, a dental condition, urinary obstruction, a prostate condition, cancer, diabetes, and chronic pain.
21. The method of feature 19 or 20, wherein adhesion of cells of the subject to the implanted medical device is enhanced by the titanium dioxide coating.
22. The method of any of features 19-21, wherein proliferation of cells of the subject on or near the implanted medical device is enhanced by the titanium dioxide coating.
23. The method of any of features 19-22, wherein growth of bacteria on or near the implanted medical device is enhanced by the titanium dioxide coating.
24. The method of any of features 19-23, wherein healing of a surgical wound is promoted by the titanium dioxide coating or the probability of post-surgical infection is reduced by the titanium dioxide coating.
25. The method of any of features 19-24, wherein the method comprises performing percutaneous coronary intervention (PCI).
26. The method of feature 25, wherein the implantable medical device is a bioresorbable vascular scaffold, and wherein restoration time following PCI is extended by the titanium dioxide coating.
27. The method of any of features 19-25, wherein the method comprises performing orthopedic surgery or a dental procedure.
28. A method of coating a surface of an implantable medical device with a titanium dioxide coating, the method comprising:

(a) providing a medical device comprising a surface to be coated;

(b) performing one cycle of atomic layer deposition to coat at least a portion of the surface with a first atomic layer of titanium dioxide; and

(c) performing one or more additional cycles of atomic layer deposition to coat the first atomic layer of titanium dioxide one or more additional atomic layers of titanium dioxide.

29. The method of feature 28, wherein each atomic layer of titanium dioxide has a thickness of about 0.4 angstrom.
30. The method of feature 28 or 29, wherein the coating comprises amorphous titanium dioxide.
31. The method of any of features 28-30, wherein the atomic layer deposition is carried out at a temperature in the range from about 130° C. to about 165° C., or from about 145° C. to about 155° C.
32. The method of any of features 28-30, wherein each cycle of atomic layer deposition comprises:
(i) exposing a surface to be coated to tetrakis(dimethylamido)titanium (TDMATi) gas in a reaction chamber;
(ii) purging the chamber with an inert gas;
(iii) exposing the coating to H₂O; and
(iv) purging the chamber again with an inert gas.
   33. The method of feature 32, wherein the exposure to tetrakis(dimethylamido)titanium is performed for about 100 milliseconds.
   34. The method of feature 32 or 33, wherein the exposure to H₂O is performed for about 100 milliseconds.
   35. The method of any of features 28-34, wherein the surface to be coated comprises a metal or metal alloy.
   36. The method of feature 35, wherein the metal or metal alloy is selected from the group consisting of Mg—Zn, Ti—V—Al, Ti, and Mg.
   37. The method of any of features 28-36, wherein a total of about 600 to about 3250 cycles of atomic layer deposition are performed.
   38. The method of feature 37, wherein the total thickness of the titanium dioxide coating is from about 24 nm to about 130 nm.
   39. A kit for implanting a coated medical device, the kit comprising the implantable medical device of any of features 1-18 and instructions for use of the device.
   40. The kit of feature 39 comprising a plurality of said implantable medical devices, the plurality of devices having a range of different sizes.
   41. The kit of feature 39 of 40, wherein contents of the kit are packaged and sterile.
   42. The kit of any of features 39-41, wherein the kit comprises one or more bioresorbable vascular scaffolds for percutaneous coronary intervention, instructions for use, and optionally one or more further devices for use in performing said percutaneous coronary intervention.

### BRIEF DESCRIPTION OF THE DRAWINGS

FIG. 1 shows a schematic illustration of an example of an atomic layer deposition (ALD) process using tetrakis(dimethylamido)titanium (TDMATi) and H₂O to coat a Mg—Zn substrate with a nanoscale thickness TiO₂ film [19].

FIG. 2 shows a schematic illustration of an example of a viscous flow ALD reactor designed for coating flat samples [22]. The dashed arrows indicate the flow across samples. The reference numerals refer to: ALD chamber (1), heated stage (2), inlet (3), outlet (4), carrier gas flow (e.g., N₂) (5), flow to vacuum pump (6), precursor (7), and oxidant (8).

FIG. 3A shows a scanning electron microscope image of Mg—Zn control, uncoated alloy; scale bar is 200 nm. FIG. 3B shows a scanning electron microscope image of Mg—Zn—TiO₂, (TiO₂ deposition at 150° C.); scale bar is 200 nm. FIG. 3C shows a scanning electron microscope image of Mg—Zn—TiO₂ (TiO₂ deposition at 200° C.); scale bar is 200 nm.

FIG. 4A shows atomic force microscopy (AFM) and RMS roughness of Mg—Zn control, uncoated alloy. FIG. 4B shows AFM and RMS roughness of Mg—Zn—TiO₂ (TiO₂ deposition at 150° C.). FIG. 4C shows AFM and RMS roughness of Mg—Zn—TiO₂ (TiO₂ deposition at 200° C.).

FIG. 5A shows X-ray photoelectron spectroscopy (XPS) graphs for titanium scan of Mg—Zn control alloy (no TiO₂), Mg—Zn—TiO₂ coating at 150° C., and Mg—Zn—TiO₂ coating at 200° C., without soak in medium. FIG. 5B shows X-ray photoelectron spectroscopy (XPS) graphs for titanium scan of Mg—Zn alloy control (no TiO₂), Mg—Zn—TiO₂ coating at 150° C., and Mg—Zn—TiO₂ coating at 200° C., with 3-day soak in medium.

FIG. 6 shows the X-ray diffraction (XRD) patterns of Mg—Zn alloy control, Mg—Zn—TiO₂ coating at 150° C., and Mg—Zn—TiO₂ coating at 200° C.

FIG. 7 shows water contact angle measurements on Mg—Zn alloy control samples, Mg—Zn—TiO₂ (coating at 150° C.) samples, and Mg—Zn—TiO₂ (coating at 200° C.) samples. Data represents mean±standard deviation, N=3; **p<0.01; ***p<0.001 compared with control.

FIG. 8 shows the amount of adsorbed bovine serum albumin protein on sample surfaces after 24 hours of culture in a 0.01% BSA solution, N=2; data represents mean±standard deviation.

FIG. 9A shows a fluorescence microscope image of human coronary artery endothelial cells (HCAECs) cultured for 4 hours on Mg—Zn control alloy. FIG. 9B shows a fluorescence microscope image of HCAECs cultured for 4 hours on Mg—Zn—TiO₂ (TiO₂ deposition at 150° C.). FIG. 9C shows a fluorescence microscope image of HCAECs cultured for 4 hours on Mg—Zn—TiO₂ (TiO₂ deposition at 200° C.).

FIG. 10A shows human coronary endothelial cell proliferation on Mg—Zn alloy control and Mg—Zn—TiO₂ (TiO₂ deposition at 150° C., and TiO₂ deposition at 200° C.) samples after 7 days. Data represents mean±standard deviation, N=2; **p<0.01; ***p<0.001 compared with control. FIG. 10B shows human coronary endothelial cell proliferation on Mg—Zn alloy control and Mg—Zn—TiO₂ (TiO₂ deposition at 150° C., and TiO₂ deposition at 200° C.) samples after 14 days. Data represents mean±standard deviation, N=2; **p<0.01; ***p<0.001 compared with control.

FIG. 11 shows energy-dispersive x-ray spectroscopy data results for Mg—Zn alloy control.

FIG. 12 shows energy-dispersive x-ray spectroscopy data results for Mg—Zn—TiO₂, (TiO₂ deposition at 150° C.).

FIG. 13 shows energy-dispersive x-ray spectroscopy data results for Mg—Zn—TiO₂, (TiO₂ deposition at 200° C.).

FIG. 14 shows bacterial density vs. as-built samples. Ti1, Ti2, Ti3, Ti4, and samples treated with ALD *p<0.01, **p<0.05 compared to control.

FIG. 15A shows a SEM image of an as-built titanium-vanadium-aluminum sample with no treatment (for control). FIG. 15B shows a SEM image of an as-built titanium-vanadium-aluminum sample treated with 10N HNO₃ for 60 minutes and then annealed. FIG. 15C shows a SEM image of an as-built titanium-vanadium-aluminum sample treated with 10N HNO₃ for 90 minutes and then annealed. FIG. 15D shows a SEM image of an as-built titanium-vanadium-aluminum sample treated with 12N HNO₃ for 60 minutes and then annealed. FIG. 15E shows a SEM image of an as-built titanium-vanadium-aluminum sample treated with 12N HNO₃ for 90 minutes and then annealed.

FIG. 16A shows a higher-magnification (5000×) SEM image of an as-built titanium-vanadium-aluminum sample with no treatment (for control). FIG. 16B shows a higher-magnification (2000×) SEM image of an as-built titanium-vanadium-aluminum sample treated with 10N HNO₃ for 60 minutes and then annealed. FIG. 16C shows a higher-magnification (5000×) SEM image of an as-built titanium-vanadium-aluminum sample treated with 10N HNO₃ for 90 minutes and then annealed. FIG. 16D shows a higher-magnification (3000×) SEM image of an as-built titanium-vanadium-aluminum sample treated with 12N HNO₃ for 60 minutes and then annealed. FIG. 16E shows a higher-magnification (5000×) SEM image of an as-built titanium-vanadium-aluminum sample treated with 12N HNO₃ for 90 minutes and then annealed. FIG. 16F shows a high-magnification (3000×) SEM image of a titanium-vanadium-aluminum sample with (no treatment, for control). FIG. 16G shows a high-magnification (3000×) SEM image of a titanium-vanadium-aluminum sample with treated with 10N HNO₃ for 60 minutes and then annealed. FIG. 16H shows a high-magnification (3000×) SEM image of a titanium-vanadium-aluminum sample with treated with 10N HNO₃ for 90 minutes and then annealed. FIG. 16I shows a high-magnification (2000×) SEM image of a titanium-vanadium-aluminum sample with treated with 12N HNO₃ for 60 minutes and then annealed. FIG. 16J shows a high-magnification (5000×) SEM image of a titanium-vanadium-aluminum sample with treated with 12N HNO₃ for 90 minutes and then annealed.

FIG. 17A shows a SEM image of a titanium-vanadium-aluminum sample after ALD. FIG. 17B shows a high-magnification (3000×) SEM image of a titanium-vanadium-aluminum sample after ALD. FIG. 17C shows a high-magnification (2000×) SEM image of a titanium-vanadium-aluminum sample after ALD. FIG. 17D shows a high-magnification (5000×) SEM image of a titanium-vanadium-aluminum sample after ALD.

FIG. 18A shows sphere diameter distribution for an as-built titanium-vanadium-aluminum sample with no treatment (for control). FIG. 18B shows sphere diameter distribution for an as-built titanium-vanadium-aluminum sample treated with 10N HNO₃ for 60 minutes and then annealed. FIG. 18C shows sphere diameter distribution for an as-built titanium-vanadium-aluminum sample treated with 10N HNO₃ for 90 minutes and then annealed. FIG. 18D shows sphere diameter distribution for an as-built titanium-vanadium-aluminum sample treated with 12N HNO₃ for 60 minutes and then annealed. FIG. 18E shows sphere diameter distribution for a titanium-vanadium-aluminum sample after ALD.

FIG. 19A shows a SEM image of a titanium-vanadium-aluminum treated with sample, treated with 10N HNO₃ for 90 minutes; areas of the SEM image that were tested with SEM-EDS (energy dispersive X-Ray spectroscopy) are highlighted. FIG. 19B shows a high-magnification SEM image of a titanium-vanadium-aluminum treated with sample, treated with 10N HNO₃ for 90 minutes; an area that was tested with SEM-EDS (energy dispersive X-Ray spectroscopy) is highlighted.

FIG. 20 shows contact angles measured using glycerol and ethylene glycol for 1, as-built control (Ti control); 2, as-built Ti1 (10N HNO₃-60 min); 3, as-built Ti2 (10N HNO₃-90 min); 4, as-built Ti3 (12N HNO₃-60 min); and 5, as-built Ti4 (12N HNO₃-90 min).

FIG. 21 shows surface tension (surface energy, mN/m) for as-built control (Ti control), as-built Ti1 (10N HNO₃-60 min), as-built Ti2 (10N HNO₃-90 min), as-built Ti3 (12N HNO₃-60 min), as-built Ti4 (12N HNO₃-90 min), and Ti-ALD (25 nm).

FIG. 22 shows S. aureus growth on Ti samples with different ALD TiO₂ coatings (applied at 190° C., 160° C., and 120° C.) after 24 hours of culture. Data represent mean±SD, N=3, *p<0.05 compared with Ti control.

FIG. 23A and FIG. 23B show a magnesium alloy stent comprising a poly-L-lactide coating that is commercially available, Coronary Resorbable Magnesium Scaffold (RMS), BIOTRONIK®, Magmaris™, www.biotronik.com/en-de/products/coronary/magmaris.

### DETAILED DESCRIPTION

Described herein is technology for chemically depositing a thin and conformal TiO₂ coating of nanoscale thickness on substrates of a variety of materials including metals and metal alloys. Mg—Zn binary alloy and other substrates. The technology can be used to apply TiO₂ nanoscale films to magnesium-zinc (Mg—Zn) binary alloy as a platform for bioresorbable vascular scaffolds (BVS) or to other implantable medical devices. The coatings provided by the technology endow surfaces of implanted medical devices with improved adsorption of cells of the subject while inhibiting the growth of bacteria.

The coatings of the present technology are applied by atomic layer deposition (ALD). ALD provides a uniform, chemically-bonded, pinhole-free, and controlled thickness coating on primary surfaces. Since ALD is independent of line of sight, internal structures under surfaces can also be coated conformally. ALD has the ability to split binary reactions into two self-limiting half-reactions occurring on the substrate surface [18]. ALD reactions are self-terminating with precise thickness controlled by deposition cycles and have good reproducibility. ALD reactions are capable of delivering atomic or molecularly thin consistent layers on substrates. In addition, the surface morphology of the deposited TiO₂ film can be controlled by varying processing temperature to achieve favorable crystallinity and surface structure [30]. ALD is a precise technique ideal for production of critical medical devices. ALD, permits precise thickness control (from single atomic layer to 100 nm or greater), an extremely conformal coating, excellent large area uniformity, strong chemical bonding, and low growth temperature (50° C.-300° C.), with applicability to biocompatible materials (e.g., Mg—Zn Alloy). ALD can enhance surface hydrophilicity, increasing surface energy and antimicrobial properties.

An example of an ALD method for applying TiO₂ coatings to medical or other implantable devices (i.e, devices implantable in the body of a human or other mammal) utilizes a precursor of TDMATi, an H₂O oxidant, and an inert purging gas (e.g., nitrogen). For example, in a single ALD cycle a 0.1 s exposure to TDMATi, 10 s of N₂ purge, 0.015 s exposure to H₂O, and 10 s of N₂ purge can be utilized, resulting in a coating thickness of about 0.4 angstrom per cycle. After 2500 cycles the coating thickness is about 100 nm of TiO₂. The thickness can be adjusted by changing pressure, temperature, substrate composition, or selection of reactant, consistent with desired outcome. As examples, the exposure to TDMATi can be about 0.05 s, about 0.1 s, or about 0.5 s. The exposure to H₂O can be about 0.005 s, about 0.01 s, about 0.015 s, about 0.02 s, about 0.03 s, or about 0.04 s. Examples of inert gases that can be utilized include, but are not limited to, gases comprising helium (He), radon (Rd), neon (Ne), argon (Ar), xenon (Xe), nitrogen (N), and combinations thereof. The exposure and purge times can be altered if different inert gases (or combinations) are utilized.

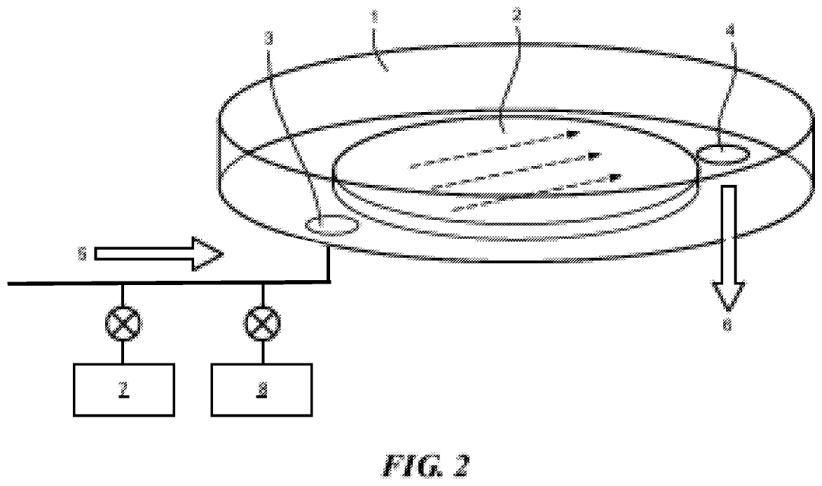

In the examples discussed below, a single ALD cycle consisted of 0.1 s exposure to TDMATi, 10 s of N₂ purge, 0.015 s exposure to H₂O, and again 10 s of N₂ purge, which was repeated for each cycle. The total flow rate of the N₂ gas was 100 standard cubic centimeters per minute (sccm). The TiO₂ thin films were deposited using at least two different temperatures, 150° C. and 200° C. For 100 nm of the TiO₂ coatings to be applied on the Mg—Zn alloys, 2500 cycles were used to complete the recipe because 0.4 angstrom was coated per cycle. FIG. 2 provides an illustration of an ALD reaction chamber.

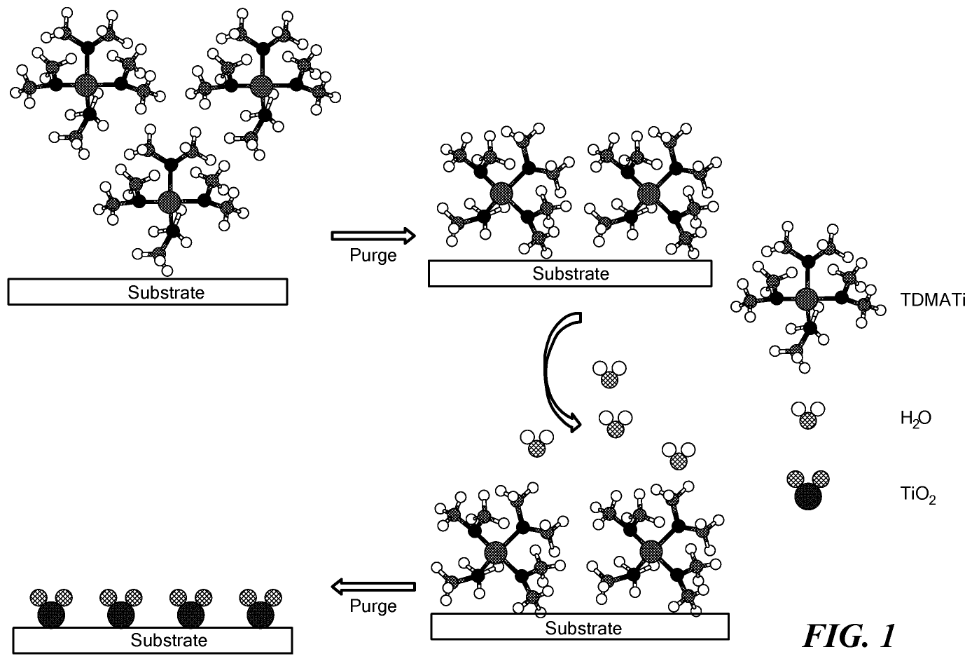

ALD can be applied to a variety of different surfaces to allow TiO₂ film growth, e.g. on flat or rough surfaces. It has been reported that crystal structures can appear when TiO₂ film growth temperatures reach above 165° C. [15]. To enable and test ALD for BVS applications, magnesium alloy (ZK61M) plates (1 mm thickness) were customized to only include Mg and Zn without any impurities (samples were purchased from Kaiqi Mold Steel Ltd., Dongguan, China). The ALD instrument was sponsored by Ultratech, Inc. (Waltham, Mass.). Mg—Zn alloy samples were cut into identical pieces (0.5 inch×0.5 inch). Samples were cleaned with 100% isopropyl alcohol (IPA) and 70% ethanol for 20 minutes, respectively. Then, the samples were dried at 100° C. inside an oven for 10 minutes. The cleaned samples were placed into a preheated ALD chamber (e.g., FIG. 2). A vacuum pump was used to create a vacuum inside the reaction chamber (for example, see FIG. 2 number 6). Titanium dioxide (TiO₂) thin films were deposited onto the Mg—Zn substrates using TDMATi and H₂O as ALD precursors (FIG. 1). Nitrogen gas served as a purging gas fed to the chamber during the entire coating process (FIG. 2, number 5). The example method (above) was repeated 2500 cycles.

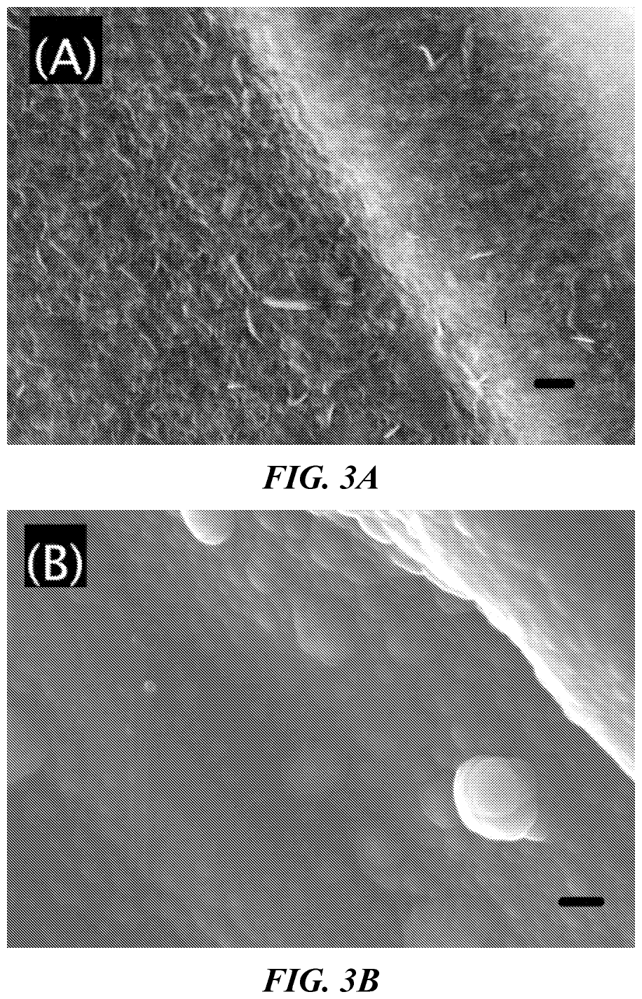

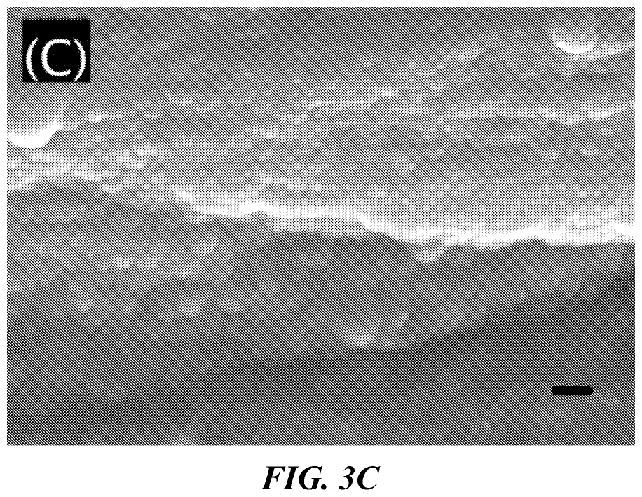

The surface morphology of the Mg—Zn alloy control (FIG. 3A) and ALD-treated Mg—Zn alloy (150° C. and 200° C.) was visualized by SEM. FIG. 3B shows SEM of the 150° C. ALD-treated Mg—Zn alloy. FIG. 3C shows SEM of the 200° C. ALD-treated Mg—Zn alloy. The black scale bar in the lower right of FIGS. 3A-3C represents 200 nm. It was shown that TiO₂ thin films coated by ALD onto Mg—Zn alloy surfaces remarkably changed surface structures. Agglomeration appeared intensively with an increase in temperature from 150° C. to 200° C. Crystallites formed on the thin film surfaces can be observed with an ALD temperature at 200° C. (FIG. 3C) compared to ALD coating at 150° C. (FIG. 3B).

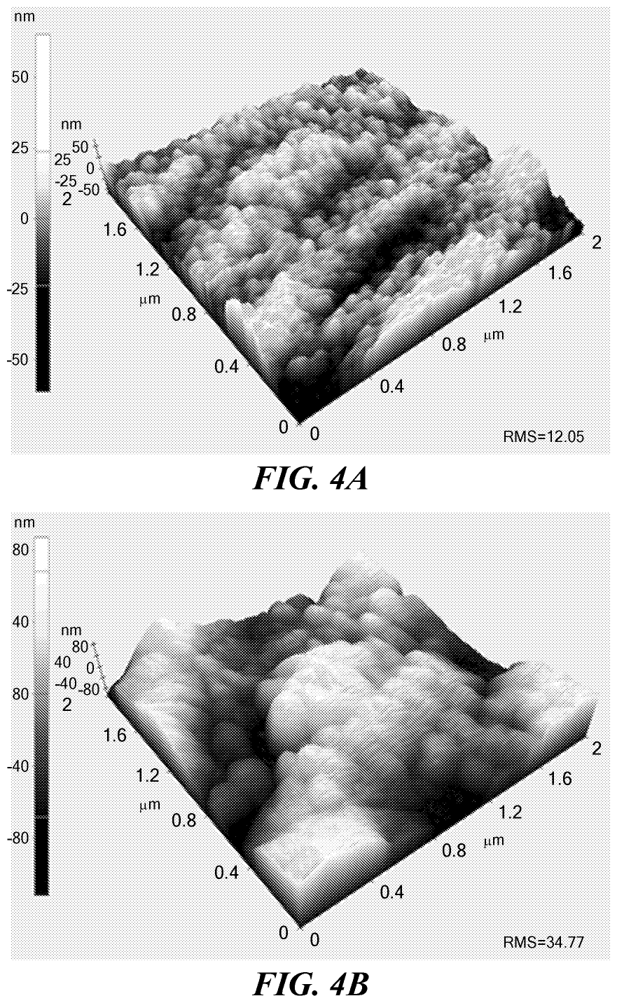

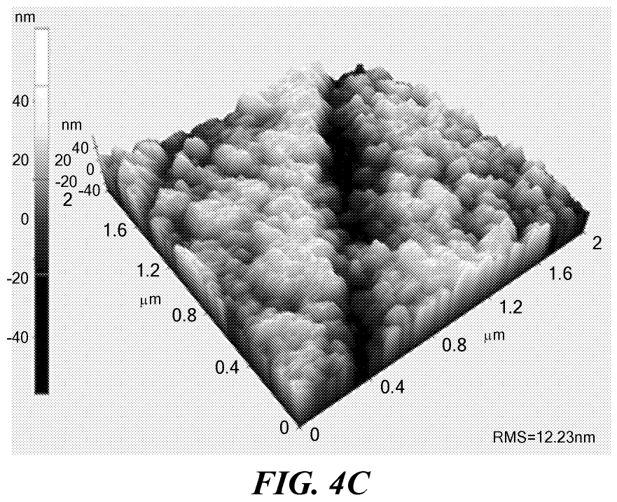

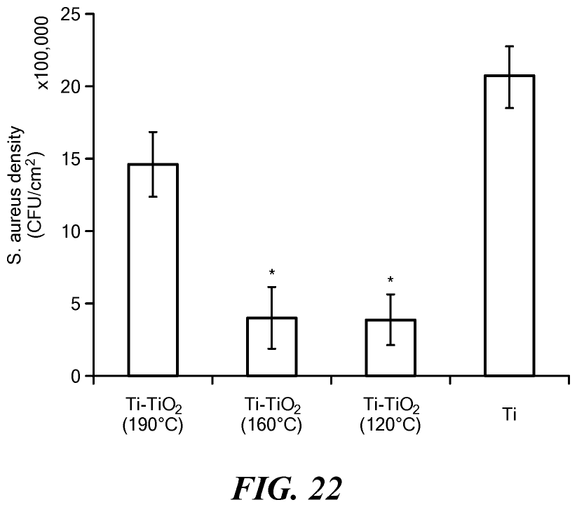

Atomic force microscopy (AFM) was performed to visualize surface topography and measure surface roughness of each sample (3D surface topography). The RMS roughness results showed an increase of surface roughness from 12.05 nm (Mg—Zn control, FIG. 4A) to 34.77 nm (Mg—Zn—TiO₂-150° C., FIG. 4B). However, TiO₂ coated at 200° C. did not change surface roughness (12.23 nm, FIG. 4C).

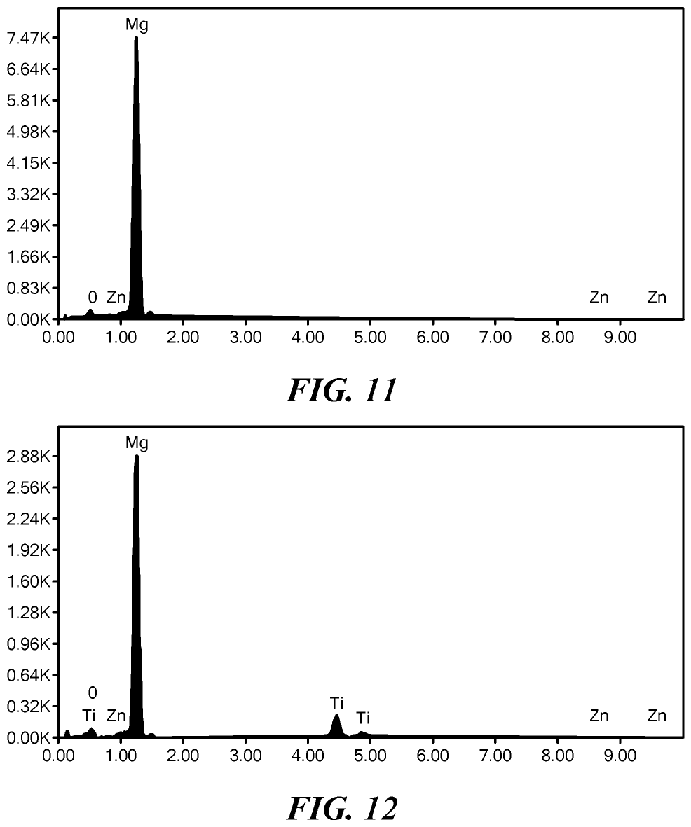

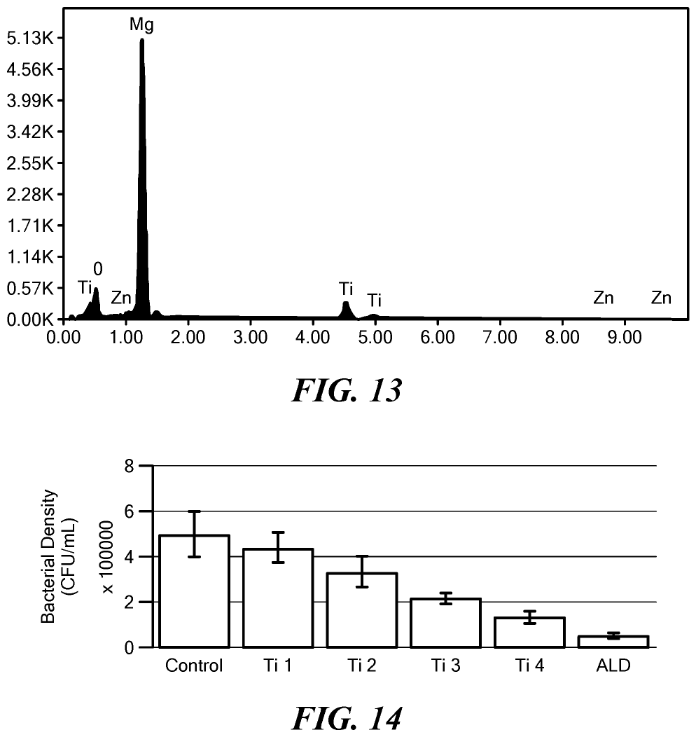

The elemental concentration of each SEM tested sample was determined by EDAX (energy dispersive analysis X-ray spectroscopy). FIG. 11 shows EDAX data results for the Mg—Zn alloy control. FIG. 12 shows EDAX data results for Mg—Zn—TiO₂, (TiO₂ deposition at 150° C.), and FIG. 13 shows EDAX data results for Mg—Zn—TiO₂, (TiO₂ deposition at 200° C.). In Table 1, the elemental weight percentages of TiO₂ coated samples are summarized compared with the Mg—Zn alloy control. The notable increase of titanium (Ti) and oxygen (O₂) indicated the existence of TiO₂ films deposited on the substrate surface.

TABLE 1
Elemental concentrations (weight %) summary
of Mg—Zn alloy samples before and after
ALD by energy-dispersive x-ray spectroscopy.

| Samples | Mg | Zn | Ti | O |
|---|---|---|---|---|
| Mg—Zn Control | 95.13 | 1.93 | N/A | 2.94 |
| Mg—Zn—TiO₂ (150° C.) | 72.01 | 1.53 | 21.69 | 4.77 |
| Mg—Zn—TiO₂ (200° C.) | 69.91 | 1.55 | 14.47 | 14.08 |

In Table 1, different elemental percentage (w/w %) ratios of Ti to O for ALD coating with the same thickness may be caused by the crystallite structure formed by the TiO₂ coating at 200° C. TiO₂ nano-thin film coating deposited a coating temperature at 190° C. has been reported to be unstable [19].

The 3D surface topography of Mg—Zn samples (FIGS. 4A-4C) revealed a smoother surface after ALD treatment for TiO₂ coated at 150° C. However, the AFM RMS results did not show an increasing trend of surface roughness as ALD operating temperature increased. ALD treatment at 200° C. did not change the surface roughness of the substrate. This might be caused by the different TiO₂ anatase crystallites formed on the 200° C. surface which are different from the amorphous surface structure created at 150° C.

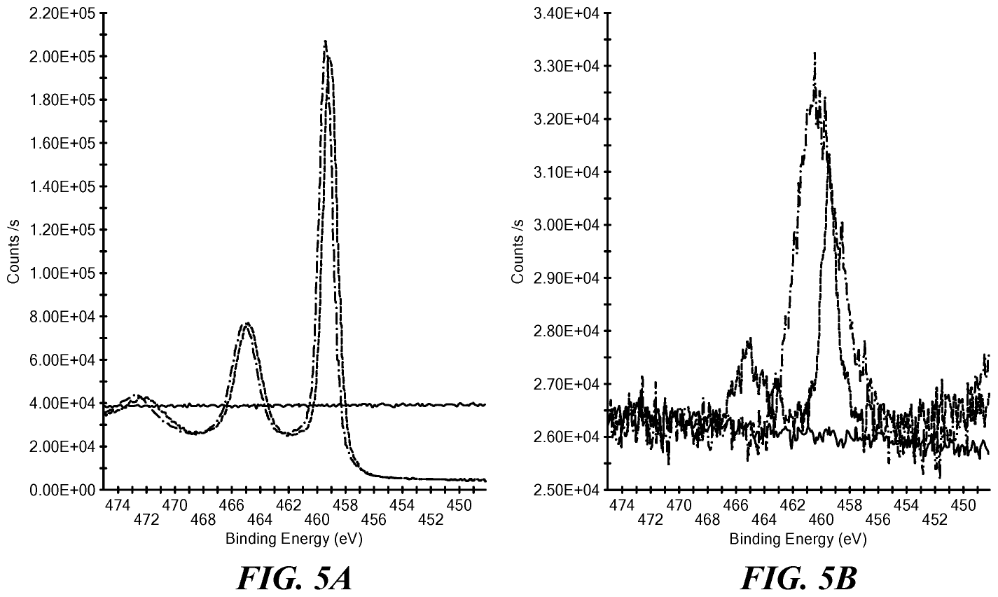

XPS graphs with titanium scans also showed the existence of TiO₂ with two peaks at 465 eV and 459 eV (FIG. 5A). In FIG. 5A, the XPS of the Mg—Zn control sample is the flat spectrum because no TiO₂ is detected. In FIG. 5A, the XPS of the 150° C. ALD and 200° C. ALD are overlaid and are similar. The XPS for all three samples was reacquired after 3 days of soaking the samples in cell medium (see FIG. 5B). In FIG. 5B, the Mg—Zn control sample remains the flat spectrum at bottom. For the 200° C. ALD coating sample, the TiO₂ thin film layer disappeared because only one peak was presented in FIG. 5B (see single large peak in top of FIG. 5B). In FIG. 5B, the sample that was TiO₂ coated at 150° C. still presented two peaks, indicating the maintenance of a TiO₂ thin film for only the 150° C. ALD coated sample.

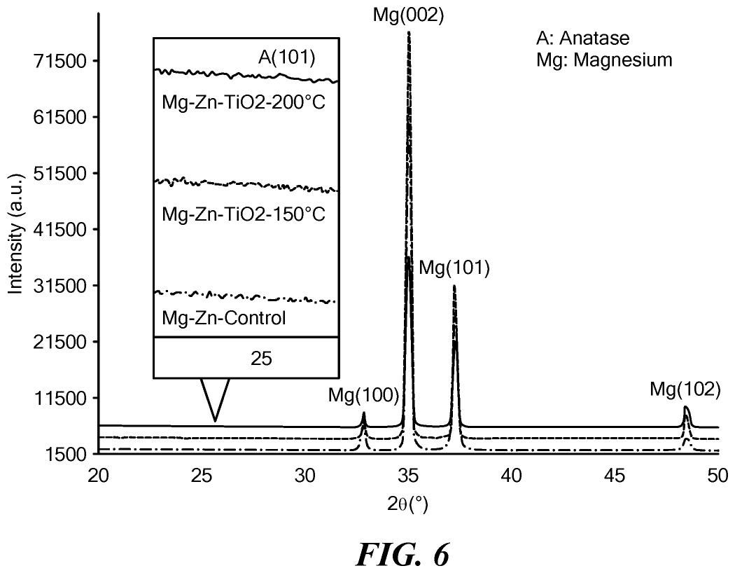

The different surface crystallinity of Mg—Zn—TiO₂ (200° C.) can be identified with the XRD analysis as shown in the XRD patterns of FIG. 6. In the enlarged inset in FIG. 6, a peak representing TiO₂ anatase appeared at 20=25.7°, which was consistent as previously reported with the formation of anatase crystallites on the surface of the material when the TiO₂ thin film was deposited above 160° C. [22].

Surface wettability, which is determined by surface topography and chemistry, can further affect protein adsorption and, thus, cell attachment, on the substrate and therefore is one of the key factors for investigating cell activities on an implant. The surface wettability of the Mg—Zn alloy control and Mg—Zn—TiO₂ (150° C. and 200° C.) was determined from static water contact angle measurements. Hydrophobicity and hydrophilicity were determined by comparing contact angles result between samples. In FIG. 8, TiO₂ coatings on Mg—Zn alloy substrates were found to be slightly more hydrophobic than controls. Mg—Zn alloy controls were more hydrophilic with contact angles around 44.5°. TiO₂ coated at 150° C. showed a slight increase of contact angle (52.5°) compared to the control. The contact angle for 200° C. thin film coatings increased to 65° indicating that the sample was much more hydrophobic than the Mg—Zn control and those prepared at 150° C. By averaging three contact angle results, surface energy was calculated based on the Owens-Wendt method (see Table 2). The dispersive surface energy is related to van der Waals and other non-site specified interactions. The polar surface energy is associated with dipole-dipole, hydrogen bonding, and other site specified interactions. In Table 2, the total surface energies were relatively lower for the Mg—Zn alloys coated with TiO₂ compared to the Mg—Zn control surface.

TABLE 2
Summary of surface wettability and surface energy
of Mg—Zn samples with different TiO₂ coatings

| | | Surface energy | | |
|---|---|---|---|---|
| | Surface wettability | (mN/m) | | |
| Samples | (contact angle/°) | γₛ^{t} | γₛ^{p} | γₛ^{d} |
| Mg—Zn Control | 44.57 ± 0.038 | 44.66 | 28.67 | 15.98 |
| Mg—Zn—TiO₂ (150° C.) | 52.50 ± 0.014 | 39.41 | 25.31 | 14.11 |
| Mg—Zn—TiO₂ (200° C.) | 64.80 ± 0.038 | 30.96 | 19.88 | 11.08 |

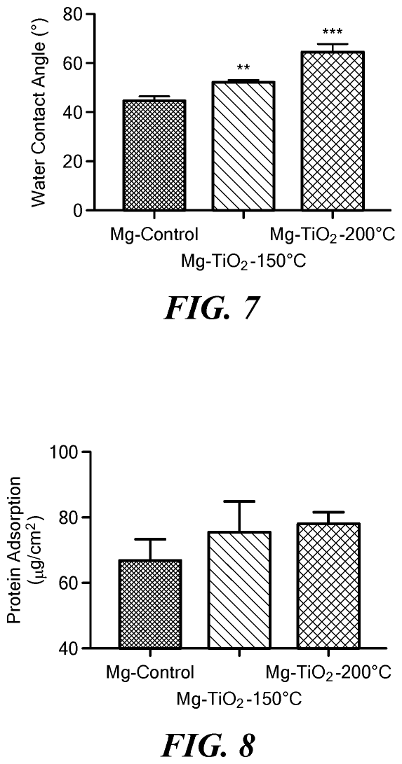

The measured water contact angles on Mg—Zn alloy control samples, Mg—Zn—TiO₂ (coating at 150° C.) samples, and Mg—Zn—TiO₂ (coating at 200° C.) samples are shown in FIG. 7. Data represents mean±standard deviation, N=3; **p<0.01; ***p<0.001 compared with control. Protein adsorption on the biomaterial surface is the initial event that occurs when the BVS are implanted. The adsorbed protein layer can affect the interactions of cells with the surface and allow for downstream cellular activities such as cell adhesion and proliferation [35]. Hydrophilicity of biomaterial surfaces is one of the main factors that affect protein adsorption. It has been reported that contact angles around 55 degrees possess the optimal surface energy to improve endothelial cell attachment [36]. In the protein adsorption study, BSA was used as a model protein to evaluate the level of protein adsorption on the ALD treated Mg—Zn substrates. As FIG. 8 shows, TiO₂ nanoscale thin film grown on the Mg—Zn alloy substrates by ALD (operated at 150° C. or 200° C.) showed a slight increase in BSA (bovine serum albumin) protein adsorption. However, the level of protein adsorption on ALD treated samples showed no statistical significance compared to the untreated control. In FIG. 8 the amount of adsorbed bovine serum albumin protein on sample surfaces after 24 hours of culture in a 0.01% BSA solution is shown, N=2, and data represents mean±standard deviation.

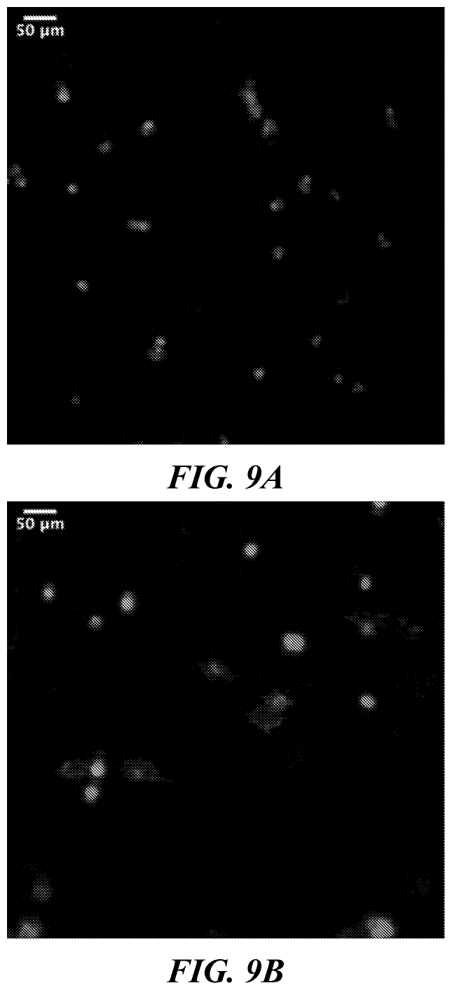

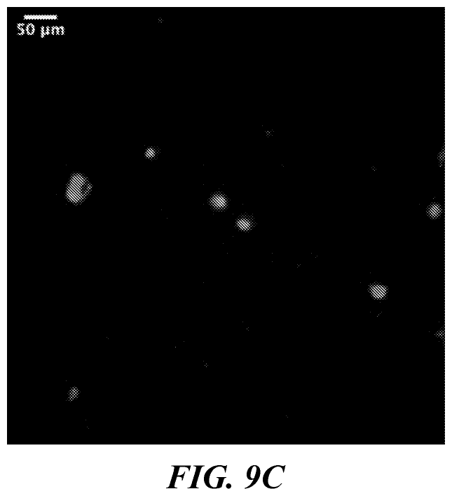

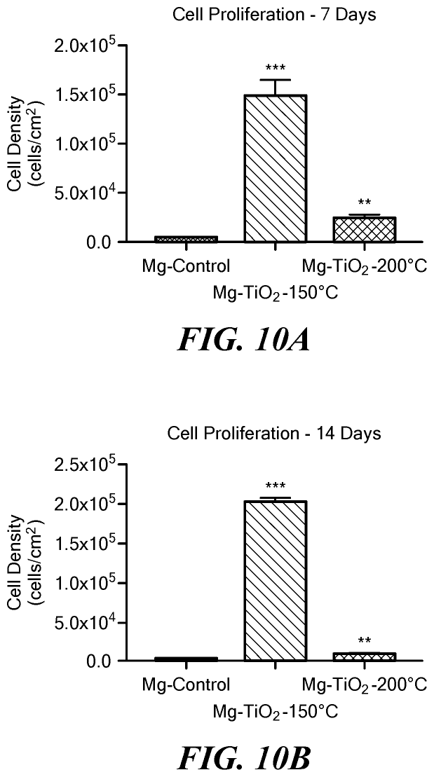

Human coronary artery endothelial cells (HCAECs, PromoCell, C-12221) were analyzed for adhesion and proliferation on the Mg—Zn alloy substrates. The fluorescence micrographs (FIGS. 9A-9C) showed that HCAECs were able to attach on all the substrates in the first 4 hours. As discussed in Example 2, the fluorescence micrographs were acquired in color to improve adhesion differentiation. The number of adhered cells on Mg—TiO₂-150° C. substrates was significantly higher than those on the control and on the Mg—TiO₂-200° C. substrates. Cells grown on Mg—TiO₂-150° C. substrates also displayed greater cell spreading and cytoskeleton development. The Mg—TiO₂-200° C. samples showed decreased cell numbers compared with the other two sample groups. However, after 7 days of cell culture, the binary control alloy and Mg—TiO₂-200° C. substrates induced very low HCAECs cell viability in vitro (FIG. 10A), and no significant increase in cell density was observed after 14 days of cell culture (FIG. 10B). In contrast, Mg—Zn—TiO₂ (150° C.) samples resulted in pronounced proliferation of HCAECs over 7 days of cell culture with a cell density at 1.5×10⁵ cells/cm². After 14 days of cell proliferation, HCAECs cell density grew even higher (2.0×10⁵ cells/cm²) presumably forming a desirable monolayer (FIGS. 10A-10B). On the other hand, Mg—Zn—TiO₂ (200° C.) and Mg—Zn control samples did not promote cell growth. By looking at the results from the 4-hour cell adhesion fluorescent images (FIG. 9A), although HCAECs adhered on the Mg—Zn control sample, the cellular cytoskeleton did not spread. Mg—Zn—TiO₂ (200° C.) similarly induced a less spread cell morphology (FIG. 9C).

Based on the data, it was hypothesized that an ALD treatment with an operating temperature at 150° C. can improve the cytocompatibility of the Mg—Zn substrates to HCAECs. On the contrary, although cells could attach on the untreated substrates, cell proliferation may have been inhibited by toxic substances generated by Mg degradation as a result of extended incubation time. During Mg degradation, one of the side products, OH⁻ ions, are generated. The release of OH⁻ ions may exhaust the physiological buffering system and cause further tissue necrosis which results in cell death or changes in cell activities due to alkalinization. This could be the reason for the low HCAECs viability on the untreated Mg—Zn control. In addition, greater hydrophobicity of the Mg—Zn—TiO₂ (200° C.) samples with a different surface structure compared with Mg—Zn—TiO₂ (150° C.) can be unfavorable for cell growth, which showed a decreased in HCAECs density through 7-14 days of cell proliferation (FIGS. 10A-10B).

Even though the TiO₂ thin films coated on Mg—Zn alloys were slightly more hydrophobic than the untreated substrates, the Mg—Zn—TiO₂ (150° C.) sample promoted cell adhesion and proliferation, indicating their potential to be a suitable BVS platform. On the other hand, Mg—Zn—TiO₂ (200° C.) with the same TiO₂ thin film coating thickness (100 nm) but different surface morphology was found not suitable for stent materials since it is unfavorable for cell adhesion and proliferation. After examining data from other alloys (below), examples of the thickness range of the ALD TiO₂ coatings herein can be about 0.4 anstrom to about 200 nm, about 10 nm to about 150 nm, about 20 nm to about 140 nm, about 30 nm to about 130 nm, about 40 nm to about 130 nm, about 50 nm to about 130 nm, about 70 nm to about 130 nm, and optionally about 100 nm. A 0.4 angstrom layer (single atomic or molecular layer) could be applicable because of the precise uniformity of ALD.

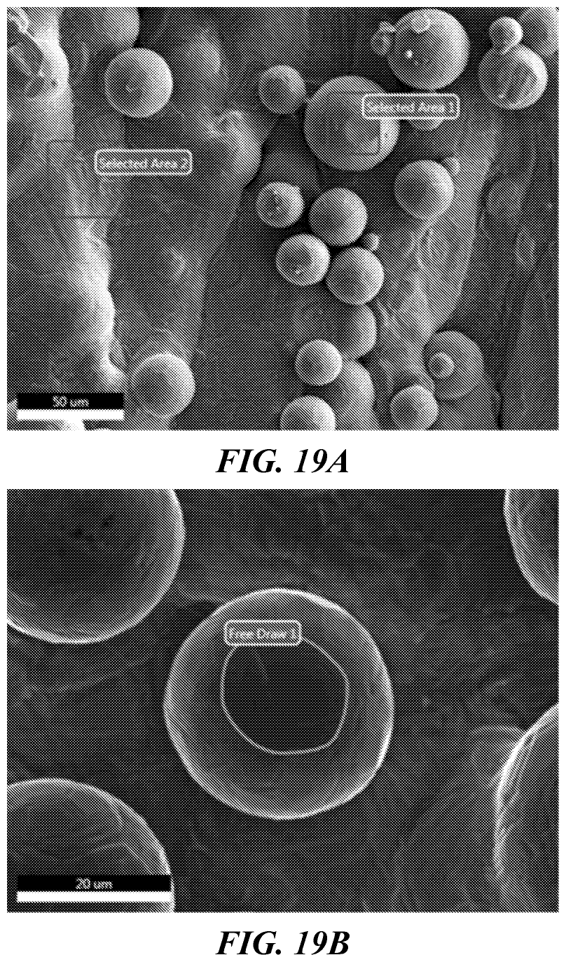

TiO₂ coating can be applied by ALD on materials other than Mg—Zn alloys. Titanium-vanadium-aluminum alloys were also examined. FIG. 19B shows a high-magnification SEM image of a titanium-vanadium-aluminum sample treated with 10N HNO₃ for 90 minutes then tested by SEM-EDS (SEM-energy dispersive X-Ray spectroscopy). Lower magnification is in FIG. 19A. The scale bar at the lower left of FIG. 19B is 20 microns. In FIG. 19B, an area that was tested with SEM-EDS is highlighted, and in FIG. 19A areas tested (EDS) are also highlighted. The EDS results for titanium showed 89.56% (w/w) and 86.19% (atomic %), (% error=2.03); EDS results for vanadium showed 5.01% (w/w) and 4.53% (atomic %), (% error=5.26); and EDS results for aluminum showed 5.43% (w/w) and 9.28% (atomic %), (% error=6.49).

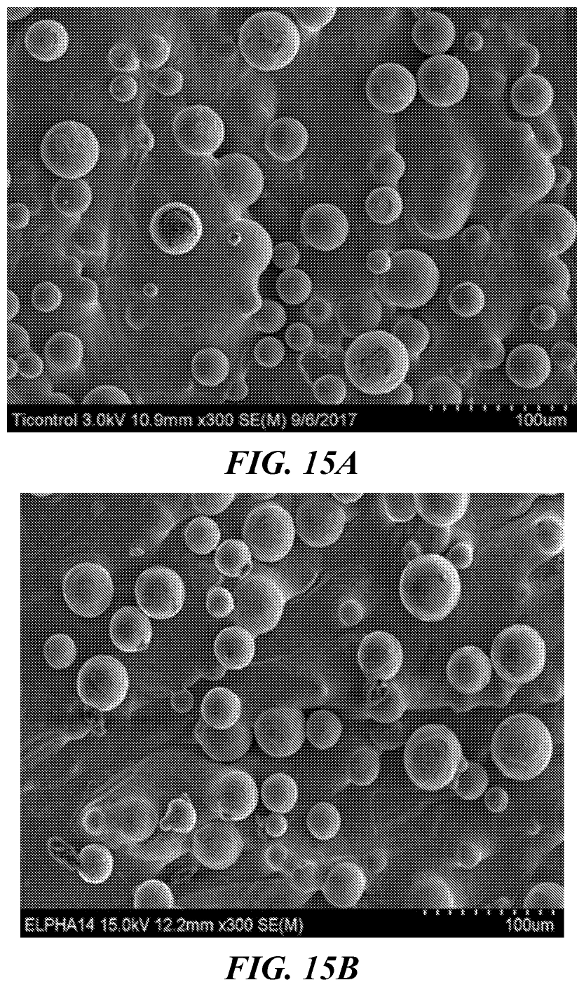

The titanium-vanadium-aluminum samples were studied for antibacterial properties (Staph. aureus density) before treatment with HNO₃ and after an ALD TiO₂ coating. ALD showed antibacterial properties compared to the Ti—V—Al control sample and compared to control samples that had been treated with HNO₃ at increasing concentrations and for increasing times (FIG. 14). The results shown in FIG. 14 are summarized in Table 3 below. The control sample is an untreated Ti—V—Al sample. Samples Ti1, Ti2, Ti3, and Ti4 were etched with HNO₃. Heat treatment was done after etching, with a heating rate of 15 C/min and furnace cooling to avoid micro-crack formation. All samples were kept at 400 C for 1 hour before cooling them down. ALD was performed at 200° C. and the thickness was 25 nm for the “As-built Ti ALD (25 nm)” sample. The precursor for TiO₂ was TDMATi. See bar to the extreme right in FIG. 14, which shows bacterial density vs. as-built samples. Table 3 below summarizes data from samples control, Ti1, Ti2, Ti3, Ti4, and samples treated with ALD, *p<0.01, **p<0.05 compared to the control sample, bacterial assay (colony forming unit). The results showed that ALD treatment successfully reduced bacterial density, even more than Ti 4 (Group 4 of acid/heat treatment).

TABLE 3
Bacterial density vs. as-built Ti—V—Al samples.
Ti1, Ti2, Ti3, Ti4, and samples treated with
ALD *p < 0.01, **p < 0.05 compared to control.

| | Bacterial | | |
|---|---|---|---|
| | density | | Number |
| Sample | (1/mL) | Error (+/−) | of tests |
| As-built control (Ti control) | 5 × 10⁵ | 10 × 10⁴ | 4 |
| As-built Ti 1 (10N HNO3-60 min) | 4.4 × 10⁵ | 6.6 × 10⁴ | 4 |
| As-built Ti 2 (10N HNO3-90 min) | 3.3 × 10⁵ | 6.6 × 10⁴ | 4 |
| As-built Ti 3 (12N HNO3-60 min) | 2.2 × 10⁵ | 2.2 × 10⁴ | 4 |
| As-built Ti 4 (12N HNO3-90 min) | 1.4 × 10⁵ | 2.5 × 10⁴ | 4 |
| As-built Ti ALD (25 nm) | 0.53 × 10⁵ | 1.1 × 10⁴ | 4 |

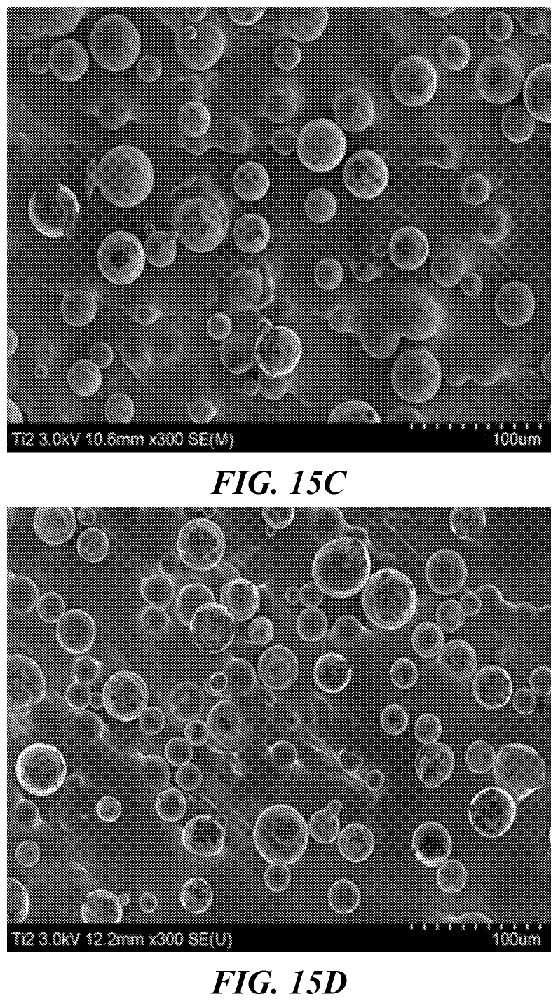

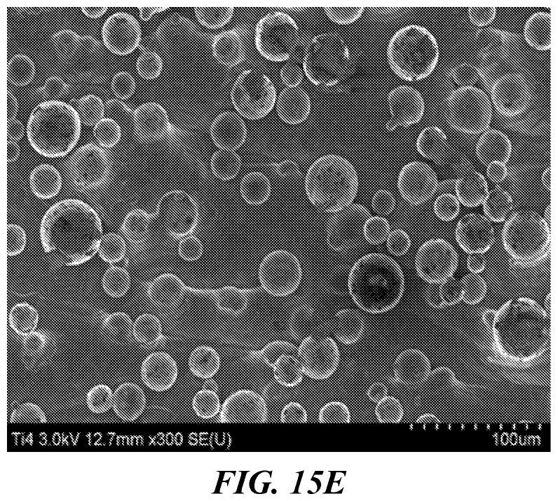

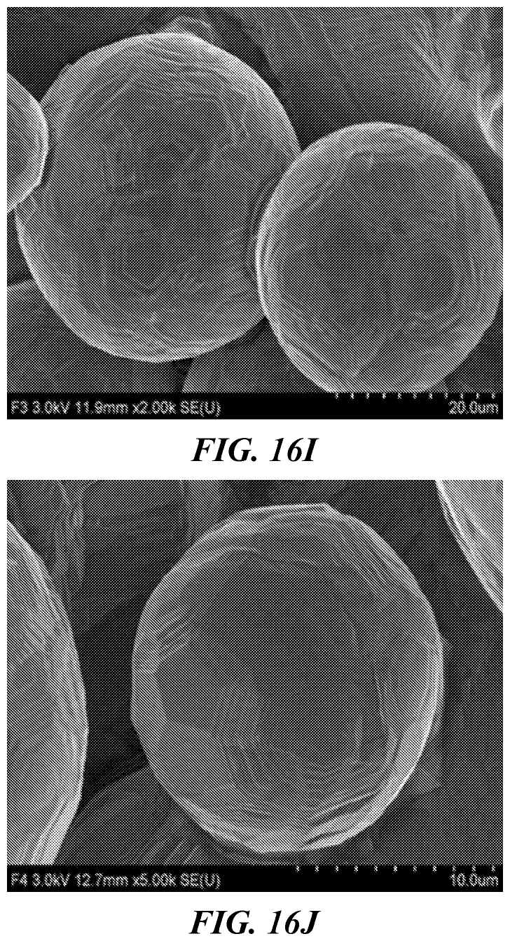

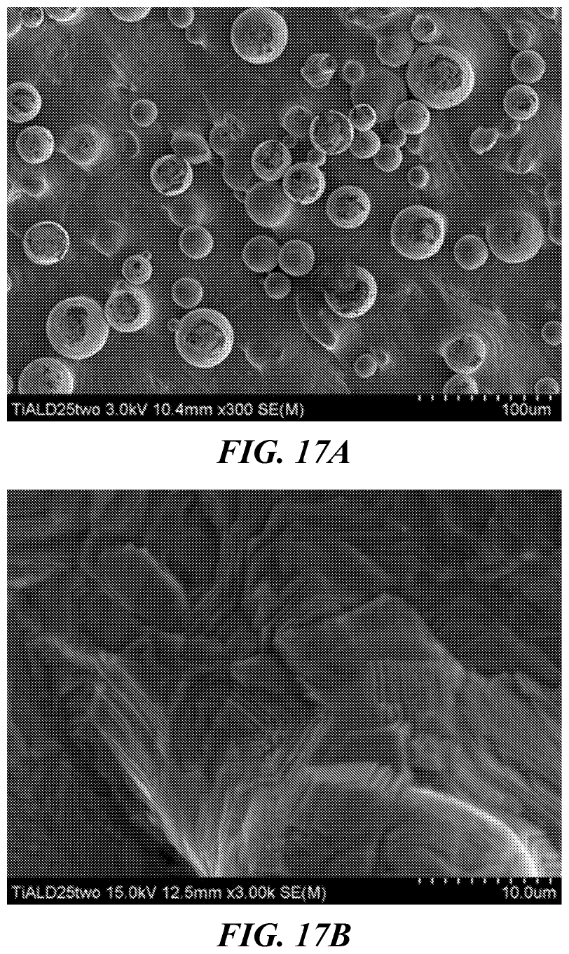

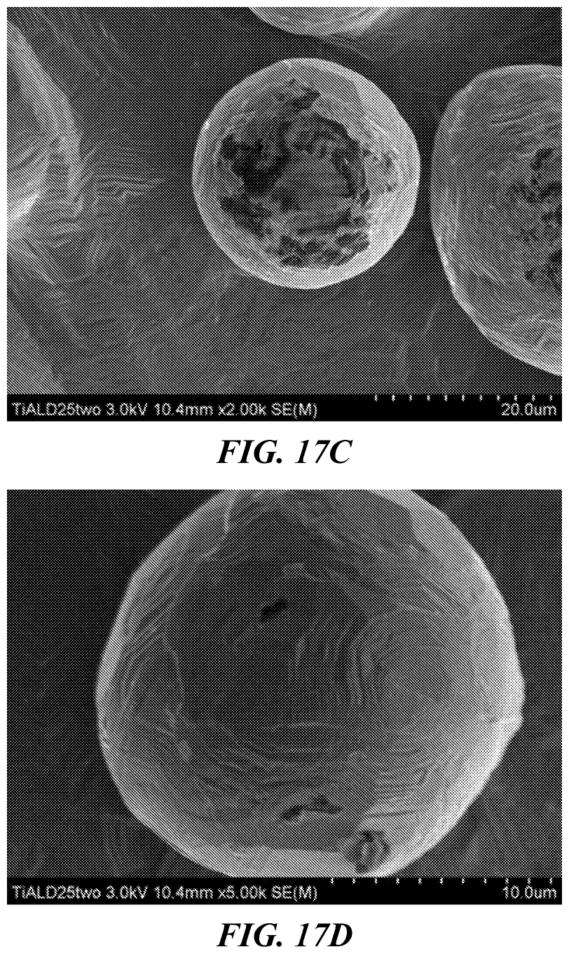

Examining the data in Table 3 above, SEM images were acquired to gain further insights into the antibacterial properties of the HNO₃ treated sample compared to the Ti ALD (25 nm) sample and the as-built (Ti—V—Al) sample. FIGS. 15A-15E are SEM images acquired at 300× for the as-built control (Ti—V—Al control) sample, as-built Ti 1 (10N HNO₃-60 min) sample, as-built Ti 2 (10N HNO₃-90 min) sample, as-built Ti 3 (12N HNO₃₋₆₀ min) sample, and the as-built Ti 4 (12N HNO₃-90 min) sample (from FIG. 14 and Table 3). FIG. 17A is an SEM image (300×) of the as-built Ti ALD (25 nm) from FIG. 14 and Table 3. In all of FIGS. 15A-15E and FIG. 17A, small spheres can be seen.

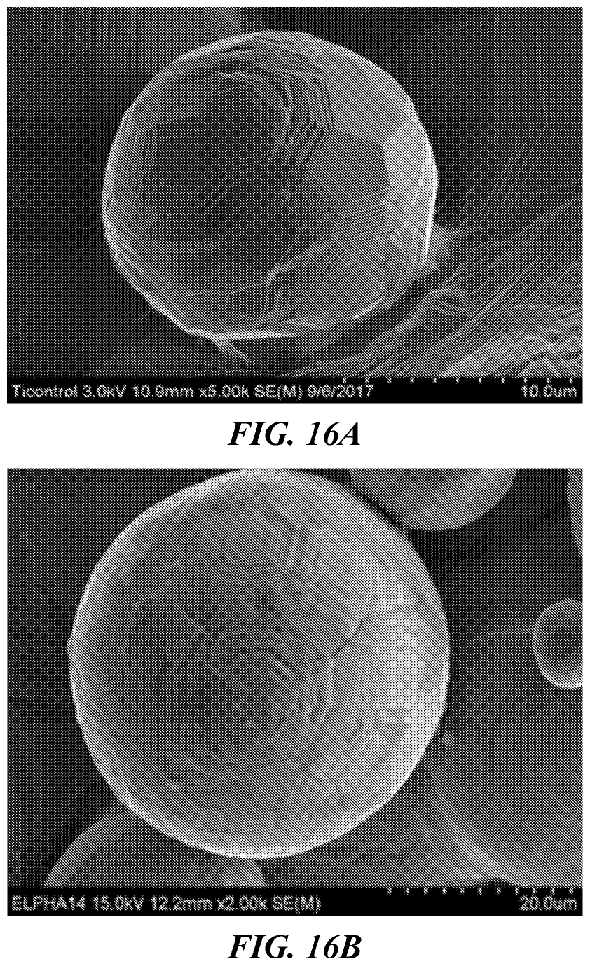

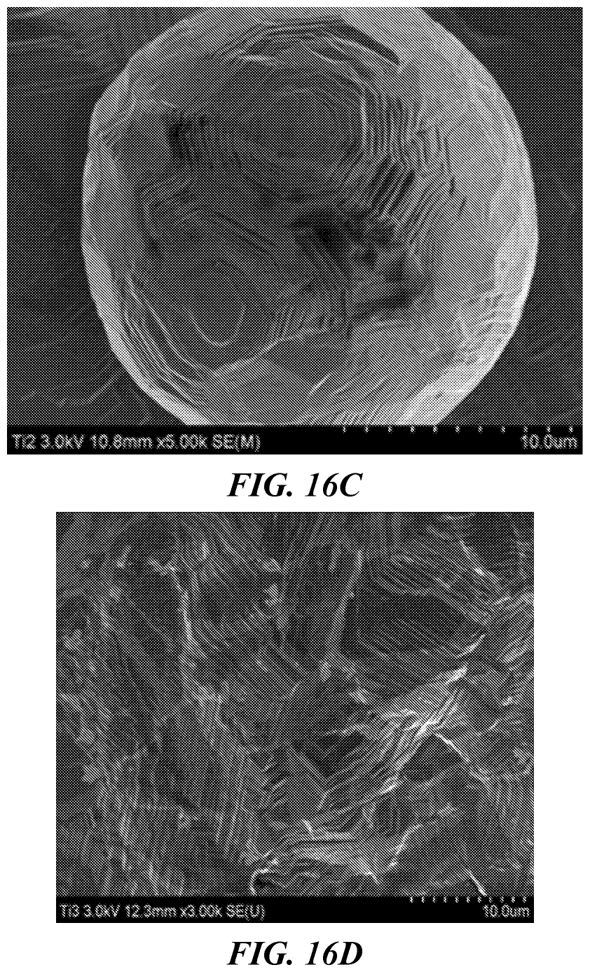

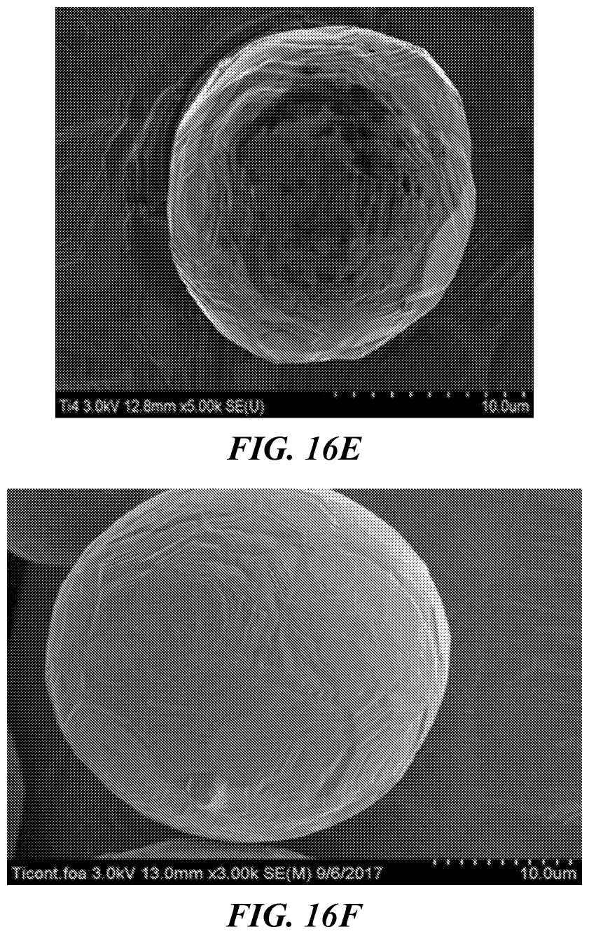

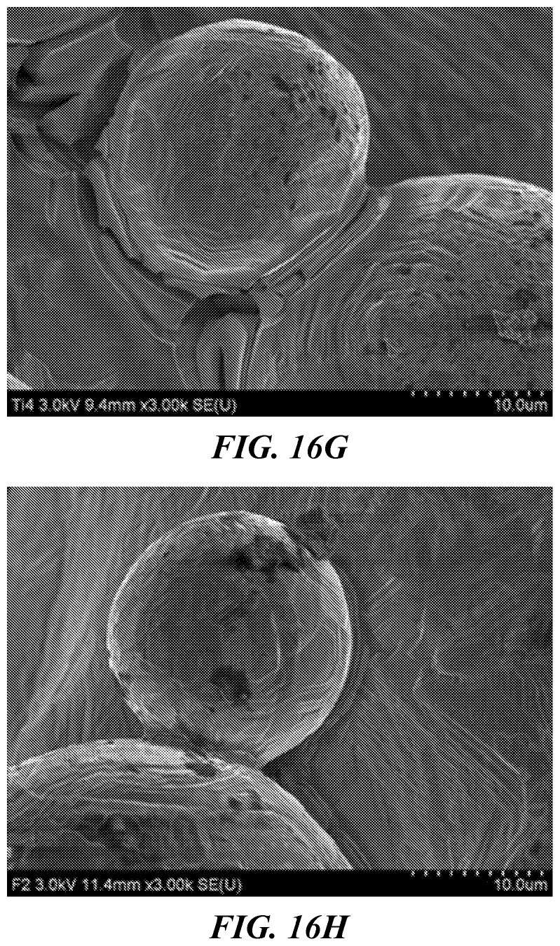

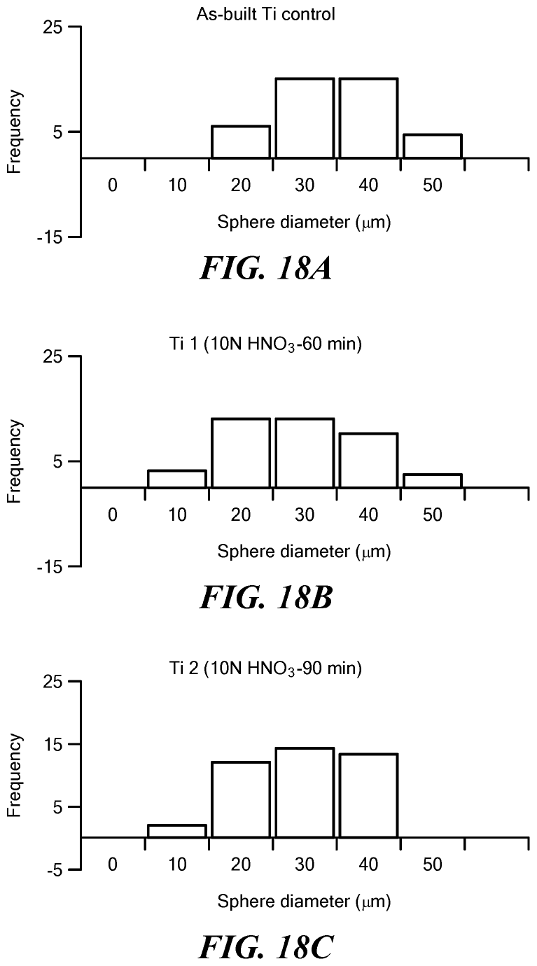

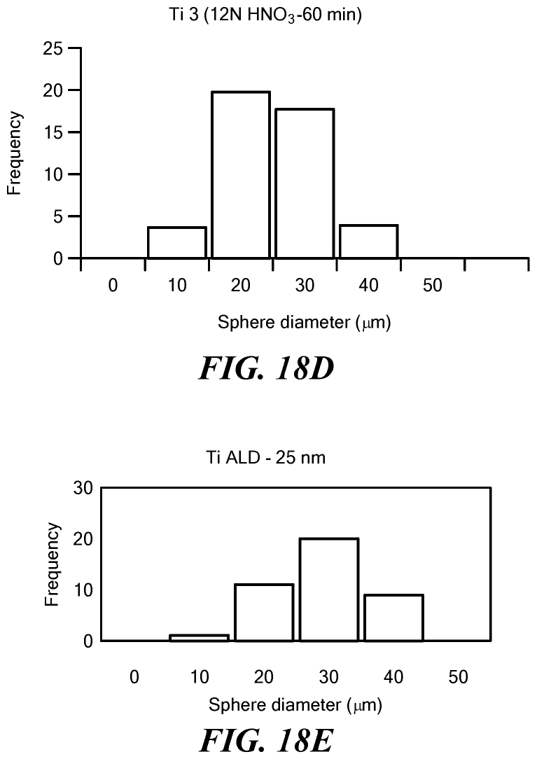

Higher magnification SEM images, from 2000× to 5000×, were acquired in FIGS. 16A and 16F for the Ti—V—Al control sample and in FIGS. 16B-16E and 16G-16J for the HNO₃ treated samples. SEM at 2000×-5000× for the as-built Ti ALD (25 nm) is shown in FIGS. 17B-17D. Size distributions of the sphere diameters are shown in histograms in FIGS. 18A-18E.

Results from SEM images with magnification of 300× indicate that the average diameter of the spheres on the surface is not significantly different. However, the distribution histograms show that as the concentration and time of acid etching is increased, the number of small sphere increases and almost all the big spheres disappear. Therefore, the antimicrobial properties may be improved due to the increased roughness of the surface (Table 4 below and FIGS. 18A-18E).

TABLE 4
Sphere diameters (mean ± S.D.)

| | Sphere diameter | |
|---|---|---|
| Sample | (μm) | S.D. (μm) |
| As-built control (Ti control) | 29.60 | 6.90 |
| As-built Ti 1 (10N HNO3-60 min) | 24.60 | 7.20 |
| As-built Ti 2 (10N HNO3-90 min) | 23.67 | 6.45 |
| As-built Ti 3 (12N HNO3-60 min) | 20.10 | 5.77 |
| As-built Ti 4 (12N HNO3-90 min) | 20.42 | 3.50 |
| Ti ALD (25 nm) | 23.90 | 6.00 |

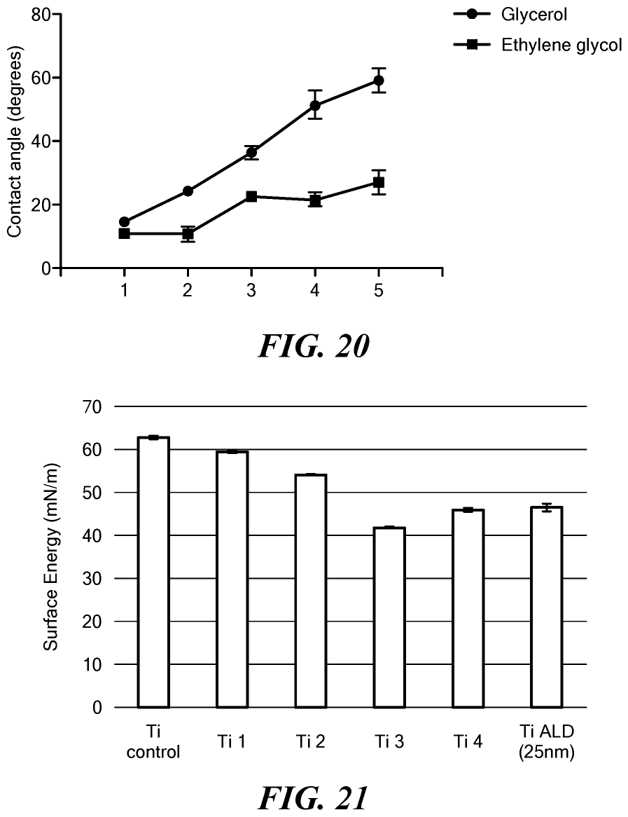

FIG. 20 shows contact angles measured using glycerol and ethylene glycol for 1, as-built control (Ti control); 2, as-built Ti1 (10N HNO₃-60 min); 3, as-built Ti2 (10N HNO₃-90 min); 4, as-built Ti3 (12N HNO₃-60 min); and 5, as-built Ti4 (12N HNO₃-90 min). The Ti-ALD (25 nm) sample is shown in Table 5 below. Tables 5 and 6 summarize the data below.

TABLE 5
Contact angles using glycerol as the solvent

| | Contact angle | |
|---|---|---|
| Sample | (degrees) | Error (+/−) |
| As-built control (Ti control) | 14.8 | 0.15 |
| As-built Ti 1 (10N HNO3-60 min) | 24.5 | 0.2 |
| As-built Ti 2 (10N HNO3-90 min) | 36.5 | 2 |
| As-built Ti 3 (12N HNO3-60 min) | 51.5 | 4.45 |
| As-built Ti 4 (12N HNO3-90 min) | 59 | 3.65 |
| Ti-ALD (25 nm) | 51 | 1 |

TABLE 6
Contact angles using ethylene glycol as the solvent

| | Contact angle | |
|---|---|---|
| Sample | (degrees) | Error (+/−) |
| As-built control (Ti control) | 11 | 0.1 |
| As-built Ti 1 (10N HNO3-60 min) | 10.8 | 2.3 |
| As-built Ti 2 (10N HNO3-90 min) | 22.7 | 0.4 |
| As-built Ti 3 (12N HNO3-60 min) | 21.8 | 2 |
| As-built Ti 4 (12N HNO3-90 min) | 27.1 | 3.9 |

By increasing the etching time and acid concentration, samples behave more hydrophobically, which may be related to the nano texture of the surface, and the same conclusion is applied to the ALD samples.

The Owens-Wendt equation was used for measuring the surface tension. Contact angles were calculated using glycerol (dominantly polar solvent) and diiodomethane (dominantly dispersive solvent). In FIG. 21, surface tension (as surface energy, mN/m) for the as-built control (Ti control), as-built Ti1 (10N HNO₃-60 min), as-built Ti2 (10N HNO₃-90 min), as-built Ti3 (12N HNO₃-60 min), as-built Ti4 (12N HNO₃-90 min), and Ti-ALD (25 nm) sample is shown. The contact angles and surface tensions are reported in the tables below.

$${( {Y}_{S}^{D} {Y}_{I}^{D} )}^{1 / 2} + {( {Y}_{S}^{P} {Y}_{I}^{P} )}^{1 / 2} = {Y}_{I} ( cos θ + 1 ) / 2. \qquad Eq . 1$$

TABLE 7
Contact angles using diiodomethane as the solvent

| | Contact angle | |
|---|---|---|
| Sample | (degrees) | Error (+/−) |
| As-built control (Ti control) | 37.9 | 4.2 |
| As-built Ti 1 (10N HNO3-60 min) | 40 | 3.45 |
| As-built Ti 2 (10N HNO3-90 min) | 40.8 | 1.1 |
| As-built Ti 3 (12N HNO3-60 min) | 35 | 3.55 |
| As-built Ti 4 (12N HNO3-90 min) | 33.85 | 5 |
| Ti-ALD (25 nm) | 22 | 5 |

TABLE 8
SurfaceTensions

| | Surface tension | |
|---|---|---|
| Sample | (mN/m) | Error (+/−) |
| As-built control (Ti control) | 62.75 | 0.4 |
| As-built Ti 1 (10N HNO3-60 min) | 59.5 | 0.3 |
| As-built Ti 2 (10N HNO3-90 min) | 54.2 | 0.1 |
| As-built Ti 3 (12N HNO3-60 min) | 42 | 0.1 |
| As-built Ti 4 (12N HNO3-90 min) | 46 | 0.4 |
| Ti-ALD (25 nm) | 46.5 | 1 |

For comparison, the antibacterial effect of different TiO₂ coatings, ALD applied at 190° C., 160° C., and 120° C., are compared with a Ti—V—Al control in FIG. 25, which shows S. aureus growth on the samples after 24 hours of culture. Data represent mean±SD, N=3. *p<0.05 compared with Ti—V—Al control. In summary, the of surface wettability and surface energy of Ti—V—Al samples with different TiO₂ coatings showed slightly more hydrophilic, total surface energy higher than control, and antimicrobial properties.

Increased protein adsorption might play an important role in inhibiting bacteria adhesion and growth. Casein is found in the culture medium. Those proteins could interact with bacteria cell membranes and prevent bacteria cells from attaching to the surface. The ideal surface energy for protein adsorption that may decrease the bacterial growth on the implant surface is reported as 42.5 mN/m. Based on Khang's equation, which relates surface energy and roughness, and also the findings in other studies which show the value of the constants in Khang's equation (ρ and E_{o,s}) we can calculate the optimum required roughness on the titanium implants' surface which adsorbs protein and inhibits bacteria growth. According to ideal surface energy (Eₛ(RMS_{eff})=42.5 mN/m), the roughness should be ˜ 40 nm. The roughness can be about 20 nm to about 75 nm, about 25 nm to about 65 nm, about 30 nm to about 60 nm, about 35 nm to about 55 nm, about 35 nm to about 50 nm, or about 35 nm to about 45 nm.

Eₛ(RMS_{eff})=ρ×RMS_{eff}+E_{o,s} Eq. 2.

In Table 8 above, samples As-built Ti 3 (12N HNO3-60 min), As-built Ti 4 (12N HNO3-90 min), and Ti-ALD (25 nm) have the surface energies 42 mN/m, 46 mN/m, and 46.5 mN/m, respectively, very close to the ideal value. It demonstrates that the surface energy for the aforementioned samples are in the ideal range that can inhibit the bacteria growth on the surface by adsorbing a layer of protein that can interact with the bacteria membrane.

By etching or roughening the surface of a Ti—V—Al alloy, Ti metal, or other material substrate (and optionally annealing) before utilizing ALD to apply TiO₂ coatings, the roughness, spheres, or texture of a substrate's surface can be modified before ALD. Sandblasting can also modify surface roughness before ALD. An example of etching is to apply 10N to 12N HNO₃ to a material substrate (e.g., Ti, Ti—V—Al, or other metals) for about 50 to 100 minutes. The HNO₃ can be a foam if needed to improve surface uniformity/adhesion. After the etching, the HNO₃ can be rinsed from the material's surface. The material can then be annealed at about 400° C. for about 1 hour, and the material is cooled. Heat treatment can be done after etching with a heating rate of about 15° C./min and furnace cooling to avoid any micro-crack formation. Samples can be kept at 400° C. for 1 hour before cooling them down. The concentrations of acid, the type of acid, etching time, annealing temperature and time can be changed depending on the material of the substrate and the desired surface roughness. If sandblasting is utilized, sandblasting conditions can be changed depending on the substrate, blasting material/size, pressure, and desired roughness. The substrate surface can be thoroughly cleaned before ALD.

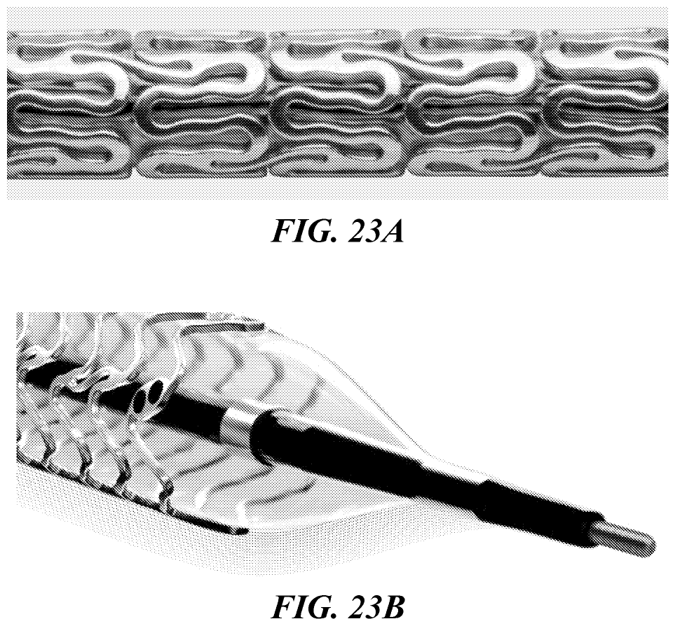

ALD can deposit nanostructure materials of a wide range of chemistry onto numerous medical devices of a wide range of chemistry. Nanoscale features of the deposited material can mimic the roughness of bone, vascular tissue, nervous system tissue, and many more. Moreover, the nanoscale features can control surface energy to dictate which proteins adsorb to increase tissue growth, decrease infection and/or inhibit inflammation. For example, (see FIGS. 23A-23B) there is currently considerable commercial interest in producing implantable biomaterials comprising magnesium-zinc (Mg—Zn) alloys; both components are completely bioresorbable within a given patient's body, with the former increasing the mechanical properties of the latter without the observation of ill-effects in vivo. The technology presented herein can provide ALD coatings for improved BVS, improved outcome from CAD, and enables a next generation of biocompatible coating. The main factor which continues to limit the broader incorporation of Mg—Zn alloys within biomedical implants is that the structures are prone to high rates of corrosion within bodily fluids, resulting in a rapid loss of structural integrity and subsequent dissolution within the implant site. There is a technical need for a well-characterized, reproducible coating which is capable of retarding the resorption of Mg—Zn alloys to manageable levels over the projected lifetime of the implanted device. ALD of titanium (IV) dioxide (TiO₂) meets this technical need, while also providing other significant advantages for biomedical implants in practice. Specifically: ALD TiO₂ coatings demonstrate an improved capability to promote mammalian cell grown and differentiation along their interfacial surfaces, thus providing increased integration of the implanted device within host tissue, while simultaneously reducing the rate of bacterial colonization along the implant surface, significantly reducing the rate of serious bacterial infection and subsequent complications following surgery.

ALD TiO₂ provides a uniform, chemically-bonded, void-free surface coating of controllable thickness which may be applied to diverse classes of basal substrates. ALD TiO₂ was initially applied to a series of Mg—Zn alloys which are commonly utilized in the construction of vascular stents, which are implemented in the clinic for various cardiovascular diseases.

The present technology provides TiO₂ coated Mg—Zn alloy substrates, produced using ALD, to serve as a BVS platform for coronary artery implantation. The TiO₂ coated substrates showed promising endothelial cell adhesion and proliferation when the film growth temperature was about 150° C. The TiO₂ nanoscale thin film acted as a protective barrier and prevented the substrates underneath the coating from interacting with surrounding biological environments. In other words, the protective layer of TiO₂ has the potential to reduce the initial degradation rate of bare Mg—Zn alloy so that the biomaterial does not lose its functionality before completion of the revascularization period (5-6 months). The ALD coating carried out at 200° C. did not show positive outcome with cell assays due to its unstable surface morphology. Crystallites formed on the surface of the coating changed its biocompatibility towards HCAECs and even killed cells. A well designed fully bioresorbable implant material should promote endothelial cell growth without additional drug elution. As a result, ALD thin film coating technology can be applied to metallic coronary stent implant materials with an optimized processing temperature control. Along the lines of the present studies, long-term simulated body fluid (SBF) simulations may be performed to see if implant functioning period values may be obtained in vitro that meet the minimum revascularization period requirement (5-6 months). ALD TiO₂ thin film coating may be further optimized to find the best processing temperature for cell promotion. Further, C-reactive protein (CRP) adsorption assays may be used to test ALD coated samples since CRP is closely related to in-stent inflammation responses which results in in-stent restenosis [38].

ALD TiO₂ coatings are poised to provide enhanced implant outcomes, based also on enhanced antimicrobial properties. Further, TiO₂ coating can be applied on materials other than Mg—Zn alloys. This is exemplified in the present disclosure by titanium-vanadium-aluminum alloys, whichwhen coated with TiO₂ deposited by ALD, show enhanced antibacterial property.

### EXAMPLES

### Example 1: Materials and Methods

### Surface Characterization

Surface morphology of the samples was characterized by scanning electron microscopy (SEM, Hitachi S-4800). The qualitative and quantitative analysis of titanium scans for samples soaked in medium for 0 and 3 days was conducted using an X-ray Photoelectron Spectroscopy (XPS, XRA008 Thermo Scientific K-alpha plus XPS System) with the data analysis software Advantage. Compositional analysis was conducted using an Energy-dispersive X-ray Spectroscopy (EDAX, Hitachi S-4800). Atomic Force Microscope (AFM; Parks Scientific XE-7 AFM) was used to measure surface roughness of ALD treated Mg—Zn samples. Each sample was analyzed under non-contact mode using a silicone ultrasharp cantilever (MikroMasch). A 2 μm×2 μm AFM field was analyzed for each sample and the scan rate was chosen to be 0.5 Hz. Image analysis software (XEI) was used to generate 3D topography images and to compare the root-mean-square (RMS) roughness of the samples obtained by the software. The crystallinity of the TiO₂ layers was investigated using an X-ray Diffractometer (XRD, Ultima, Rigaku Corp.) fitted with a Cu Kα radiation. The XRD was operated at 40 kV and 44 mA with a step width of 0.1 e and a count time of 0.5 s. The scanning range (20) of the XRD trial was 20-90°. Phase identification was performed using the standard JCPDS database. To assess sample surface wettability, water contact angles were measured using a ProScope HR Microscope at room temperature. A droplet of deionized water was added to each sample surface. Three identical samples were measured to calculate contact angle results. The average contact angle was determined, and the Owens-Wendt method [23] was used to calculate the surface free energy. See equations below.

$${γ}_{s}^{d} = \frac{{{γ}_{l}^{d} ( 1 + cos θ )}^{2}}{4} ; {γ}_{l}^{t} ( 1 + cos θ ) = 2 ( \sqrt{{γ}_{l}^{p} {γ}_{s}^{p}} + \sqrt{{γ}_{l}^{d} {γ}_{s}^{d}} ) ; {γ}_{s}^{t} = {γ}_{s}^{d} + {γ}_{s}^{p} \qquad Eq . 3$$

where, γ_{d}^{d}, γₛ^{p}, and γₛ^{t} are the dispersive, polar, and total components of the substrate surface energy; γₗ^{d}, γₗ^{p}, and γₗ^{t} are dispersive, polar, and total components of the liquid surface tension respectively; and θ is the contact angle as determined.

### Protein Adsorption Assays

Bicinchoninic acid (BCA) protein assay kit (Thermo Scientific) was used to quantify the total amount of bovine serum albumin (BSA) protein adsorbed onto the sample surfaces. 1 mg/mL (0.1%) BSA solution was prepared by diluting 30% BSA with PBS. Each sample was treated with 1 mL 0.1% BSA solution and cultured for 24 hours in an incubator (37° C., humidified, 5% CO₂). After that, BSA solution was aspirated and each sample was washed with 1 mL PBS to remove non-adsorbed proteins. Then, each sample was treated with 1 mL RIPA buffer (Sigma-Aldrich) for 10 minutes to solubilize adsorbed proteins. A working reagent (WR) was prepared using BCA protein assay kit with a 50:1 ratio of Reagent A:B. According to the BCA assay microplate protocol, the desired amount of BSA for a desired final concentration was mixed with the corresponding WR and put into a dry bath at 37° C. Finally, 200 μL of each sample of BSA was transferred to a 96-well tissue culture plate and tested at 562 nm by the plate reader (Molecular Devices, SpectraMax M3).

### Cell Assays

Cell culture: Human Coronary Artery Endothelial Cells (HCAECs, PromoCell, C-12221) were used for all mammalian cell experiments. Endothelial cells were cultured in Endothelial Cell Growth Medium (PromoCell, C-22010) with an endothelial cell growth medium supplemental mix (PromoCell, C-39215) added to the growth medium. 5 mL of 1% penicillin/streptomycin (P/S; Sigma-Aldrich) was added to the Endothelial Cell Growth Medium and filtered to be stored in a 4° C. fridge. All cells were incubated in a 37° C., humidified, 5% CO₂ and 95% air environment.

### Fluorescence Microscopy Assays

Cell adhesion samples were prepared and seeded with 100,000 cells per well. After 4 hours of incubation, the samples were washed three times with PBS and then stained for fluorescence microscopy analysis. A 3.7% formaldehyde solution was used to fix cells on samples. The samples were further permeabilized with 0.1% Triton X-100 solution for 5 minutes. Rhodamine and Hoechst (Life Technologies) actin stain dyes were used to view adherent cells on each sample. Finally, the samples were turned upside down in a new 12-well plate and imaged using a Zeiss Axio Observer Z1 with Zen 2 Pro Software.

### Cell Adhesion and Proliferation Assays

To investigate with HCAECs, Mg—Zn alloy samples were placed individually into 12-well non-tissue culture plates and sterilized with UV light inside a biohazard hood for one hour. 1 mL cell medium was added to each well and incubated for one hour. Human Coronary Endothelial Cells were seeded onto each sample at a density of 10, 000 cells/cm². For cell adhesion, endothelial cells were incubated for 4 hours at 37° C., humidified 5% CO₂ atmosphere. Cell proliferation was measured at 7 days and 14 days of culture. Cell growth medium was changed every two days during proliferation period. Phosphate-buffered saline (PBS) was used to wash off dead cells and 1 mL PBS was added to each sample and aspirated before adding new growth medium. After the incubation, each sample was washed with 1 mL PBS and an MTS dye (Promega) solution at a 1:5 ratio (MTS: Medium) was prepared. Each sample was carefully transferred to a new 12-well tissue culture plates with 1.2 mL MTS solution added into each well. Next, 12-well tissue culture plates were covered with aluminum foils and cultured for another 4 hours to allow complete reaction of the MTS dye with the metabolic products of the adherent cells. Then 100 μL of the reacted solution from each well was transferred to a 96-well tissue culture plate in triplicate. Finally, cell density data was determined from the absorbance measured by a plate reader (Molecular Devices, SpectraMax M3) at 490 nm. Standard curves for cell density calculations were utilized.

### Statistics

All cell studies were conducted in triplicate and repeated at least two times. Data were collected, and the significant differences were assessed with the probability associated with one way ANOVA tests only comparing with control data. Statistical significance was determined based on p-value being less than 0.05.

### Example 2: Characterization of TiO₂ Coated Mn—Zn Alloy Substrate

The notable increase of titanium (Ti) and oxygen (O₂) indicated the existence of TiO₂ films deposited on the substrate surface. AFM (atomic force microscopy) was performed to visualize surface topography and measure surface roughness of each sample. The RMS (root mean square) roughness results showed surface roughness from 12.05 nm (see FIG. 4A, Mg—Zn control) to 34.77 nm (FIG. 4B, Mg—Zn—TiO₂-150° C.) and 12.23 nm (FIG. 4C, Mg—Zn—TiO₂-200° C.) with increasing ALD processing temperature (see FIGS. 2A-2C. FIG. 1 AFM images and RMS roughness of (A) Mg—Zn Control, (B) Mg—Zn—TiO₂ (150° C.), (C) Mg—Zn—TiO₂ (200° C.).

XPS graphs with titanium scans also showed the existence of TiO₂ with two peaks at 465 eV and 459 eV (FIG. 5A). After 3 days of soaking in cell medium (FIG. 5B), TiO₂ thin film layer disappeared since only one peak was present for the sample with a 200° C. ALD coating. TiO₂ coated at 150° C. still presented two peaks (FIG. 5B) indicating the maintenance of TiO₂ thin film.

XRD patterns of tested samples are shown in FIG. 6. X-ray diffraction peaks were observed to fit with standard JCPDS data and compared with similar Mg—Zn alloy patterns [25]. A diffraction peak at 29=25.7° for Mg—Zn—TiO₂ (200° C.) indicates the formation of TiO₂ crystalline anatase when compared with Mg—Zn—TiO₂ (150° C.) and control. Surface wettability, which is determined by surface topography and chemistry, can further affect protein adsorption on the surface of the substrate and therefore is one of the key factors for investigating cell and bacteria activities at an interface between the implant and surrounding tissue [26, 27].

### Protein Adsorption Effect

According to the results obtained from BCA protein adsorption assay (FIG. 8), ALD-coated Mg—Zn alloy samples had slightly increased protein adsorption when compared with Mg—Zn control after the treatment in 0.01% BSA protein solution for 24 hours. The rising protein adsorption could be important for cell culture and bacteria activities since proteins could interact with cell membranes and could protect surfaces from being attacked by bacteria [28]. The amount of adsorbed bovine serum albumin protein on sample surfaces after 24 hours of culture in a 0.01% BSA solution is presented in FIG. 8 (N=2; Data represents mean±standard deviation).

### Fluorescent Microscopy Assays

Fluorescent microscopy experiments employing Rhodamine/Hoechst (red/blue signals) dyes were carried out. A fluorescent microscope image of HCAECs cultured for 4 hours on Mg—Zn Control is shown in FIG. 9A. A fluorescent microscope image of HCAECs cultured for 4 hours on Mg—Zn—TiO₂ (150° C.) is shown in FIG. 9B. A fluorescent microscope image of HCAECs cultured for 4 hours on Mg—Zn—TiO₂ (200° C.) is shown in FIG. 9C. Fluorescence micrographs of HCAECs cultured for 4 hours on Mg—Zn control and Mg—Zn—TiO₂ (150° C. and 200° C.) samples showed that HCAECs will initially adhere on Mg—Zn alloy surfaces. Although FIGS. 9A-9C are presented with no color, control samples clearly showed cell adhesion on Mg—Zn with blue signals indicating cell cores stained by Rhodamine. Red signals represented cell membranes stained by Hoechst dye. Live HCAECs before cell fixation was represented by the overlay of red and blue signals (no color in FIGS. 9A-9C).

As shown in FIG. 9A yet in grayscale, without ALD coatings, HCAECs only adhered on sample surfaces but did not promote cell growth which corresponds to the reason why most of the live cells turned out to have blue signals that were larger than red signals. Samples with TiO₂ coated at 150° C. showed impressive cell growth (FIG. 9B) as the majority of the cells were covered by red signals rather than blue signals. The growth of cell membranes was indicated by the spread of cell membranes (red signals) to represent the promotion of HCAECs under fluorescent microscopes. On the other side, samples coated at 200° C. did not show positive results corresponding to cell adhesion and cell growth (FIG. 9C). These results were confirmed by the longer-term cell proliferation studies in FIGS. 10A-10B.

### Cell Assays

Human Coronary Artery Endothelial Cells (HCAECs) form important cell monolayer that lines blood vessels, maintains vascular tone, regulates hemostasis, protects blood vessel from toxic matters, and controls inflammation [29]. During PCI, expansion of coronary stent might cause damage to the monolayer of HCAECs that lines the blood vessel. Therefore, a successful coronary scaffold should have the ability to promote the growth of HCAECs in order to heal and reconstruct blood vessel. In otherwords, a promising implantable material should accelerate HCAECs growth and protect blood vessel implanted with coronary stents from inflammation, as well as balance thrombosis and clotting. Thus, the effect of nanoscale TiO₂ thin film coating deposited by ALD on HCAECs cell proliferation was investigated for Mg—Zn—TiO₂ (150° C. and 200° C.) and Mg—Zn (control) samples. As a result, after 7 days and 14 days of cell culture, the endothelial cell density for Mg—Zn—TiO₂ (150° C.) samples was found to be enormously higher than those measured for Mg—Zn controls (FIGS. 10A-10B).

However, Mg—Zn—TiO₂ (200° C.) samples did not show high promotion of HCAECs and cell density. Unfortunately, the cell density decreased over time based on a comparison of the results of 7 days and 14 days cell culture.

As used herein, the term “about” and “approximately” include values close to the stated value as understood by one of ordinary skill in the art. For example, “about” and “approximately” can refer to values within 10%, within 5%, within 1%, or within 0.5% of a stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

### REFERENCES

1. Zipes, Douglas P., et al. “Braunwald's Heart Disease: a Textbook of Cardiovascular Medicine.” Braunwald's Heart Disease: a Textbook of Cardiovascular Medicine, 2018.
2. Iqbal, Javaid, et al. “Coronary Stents: Historical Development, Current Status and Future Directions.” British MedicalBulletin, vol. 106, no. 1, 2013, pp. 193-211.
3. Garg, Scot, and Patrick W. Serruys. “Coronary Stents Current Status.” Journal Of The American College Of Cardiology, vol. 56, no. 10, 2010, pp. S1-S42.
4. Serruys, Pw, et al. “ANGIOGRAPHIC FOLLOW-UP AFTER PLACEMENT OF A SELF-EXPANDING CORONARY-ARTERY STENT.” New England Journal Of Medicine, vol. 324, no. 1, 1991, pp. 13-17.
5. Kereiakes, Dean J, et al. “Bioresorbable Vascular Scaffolds for Coronary Revascularization.” Circulation, vol. 134, no. 2, 2016, pp. 168-82.
6. “Absorb Bioresorbable Vascular Scaffold System.” Home, www.vascular.abbott/us/products/coronary-intervention/absorb-bioresorbable-scaffold-dissolving-stent.html.
7. Azzalini, Lorenzo, and Philippe L L'Allier. “Bioresorbable Vascular Scaffold Thrombosis in an All-Comer Patient Population: Single-Center Experience.” The Journal of Invasive Cardiology, vol. 27, no. 2, 2015, pp. 85-92.
8. “REal World Advanced Experience of BioResorbable ScaffolD by SMart Angioplasty Research Team (SMART REWARD)—Full Text View.” Full Text View—ClinicalTrials.gov, clinicaltrials.gov/ct2/show/NCT02601404.
9. Song, G., and Song, S. “A Possible Biodegradable Magnesium Implant Material.” Advanced Engineering Materials, vol. 9, no. 4, 2007, pp. 298-302.
10. Li, Nan, and Yufeng Zheng. “Novel Magnesium Alloys Developed for Biomedical Application: A Review.” Journal of Materials Science &Amp; Technology, vol. 29, no. 6, 2013, pp. 489-502.
11. Tapiero, and Tew. “Trace Elements in Human Physiology and Pathology: Zinc and Metallothioneins.” Biomedicine &Amp; Pharmacotherapy, vol. 57, no. 9, 2003, pp. 399-411.
12. Bowen, Patrick K., et al. “Zinc Exhibits Ideal Physiological Corrosion Behavior for Bioabsorbable Stents.” Advanced Materials, vol. 25, no. 18, 2013, pp. 2577-2582.
13. Zhang, Shaoxiang, et al. “In Vitro Degradation, Hemolysis and MC3T3-E1 Cell Adhesion of Biodegradable Mg—Zn Alloy.” Materials Science &Amp; Engineering C, vol. 29, no. 6, 2009, pp. 1907-1912.
14. Zhang, Shaoxiang, et al. “Research on an Mg—Zn Alloy as a Degradable Biomaterial.” Acta Biomaterialia, vol. 6, no. 2, 2010, pp. 626-640.
15. Kirkland, N T. “Magnesium Biomaterials: Past, Present and Future.” Corrosion Engineering, Science and Technology, vol. 47, no. 5, 2012, pp. 322-328.
16. Koch, C. F., et al. “Pulsed Laser Deposition of Hydroxyapatite Thin Films.” Materials Science &Amp; Engineering C-Biomimetic And Supramolecular Systems, vol. 27, no. 3, 2007, pp. 484-494.
17. Yang, et al. “Modification of Degradation Behavior of Magnesium Alloy by IBAD Coating of Calcium Phosphate.” Surface &Amp; Coatings Technology, vol. 202, no. 22, 2008, pp. 5733-5736.
18. Wank, Jeffrey R., et al. “Coating Fine Nickel Particles with Al 2 O 3 Utilizing an Atomic Layer Deposition-Fluidized Bed Reactor (ALD-FBR.” Journal of the American Ceramic Society, vol. 87, no. 4, 2004, pp. 762-765.
19. Liu L, et al. “Atomic Layer Deposition of Nano-TiO2 Thin Films with Enhanced Biocompatibility and Antimicrobial Activity for Orthopedic Implants.” International Journal of Nanomedicine, 2017, pp. 8711-8723.
20. Shen, G. X., et al. “Study on a Hydrophobic Nano-TiO2 Coating and Its Properties for Corrosion Protection of Metals.” Electrochimica Acta, vol. 50, no. 25, 2005, pp. 5083-5089.
21. Basiaga, Marcin, et al. “Influence of ALD Process Parameters on the Physical and Chemical Properties of the Surface of Vascular Stents.” Archives of Civil and Mechanical Engineering, vol. 17, no. 1, 2017, pp. 32-42.
22. Tzia Ming Onn, et al. “Atomic Layer Deposition on Porous Materials: Problems with Conventional Approaches to Catalyst and Fuel Cell Electrode Preparation.” Inorganics, vol. 6, no. 1, 2018, pp.
23. Owens, D. K., and R. C. Wendt. “Estimation of the Surface Free Energy of Polymers.” Journal of Applied Polymer Science, vol. 13, no. 8, 1969, pp. 1741-1747.
24. Aarik, et al. “Morphology and Structure of TiO2 Thin Films Grown by Atomic Layer Deposition.” Journal of Crystal Growth, vol. 148, no. 3, 1995, pp. 268-275.
25. Jenei, et al. “X-Ray Diffraction Study on the Microstructure of a Mg—Zn—Y Alloy Consolidated by High-Pressure Torsion.” Journal of Alloys and Compounds, vol. 539, no.

C, 2012, pp. 32-35.
26. Liu, Kesong, et al. “Bio-Inspired Titanium Dioxide Materials with Special Wettability and Their Applications.” Chemical Reviews, vol. 114, no. 19, 2014, pp. 10044-10094.
27. Xu, and Siedlecki. “Effects of Surface Wettability and Contact Time on Protein Adhesion to Biomaterial Surfaces.” Biomaterials, vol. 28, no. 22, 2007, pp. 3273-3283.
28. H. P. Felgueiras, J. C. Antunes, M. C. L. Martins, M. A. Barbosa, “1—Fundamentals of protein and cell interactions in biomaterials”, Editor(s): Mario A. Barbosa, M. Cristina L. Martins, Peptides and Proteins as Biomaterials for Tissue Regeneration and Repair, Woodhead Publishing, 2018, Pages 1-27.
29. Brutsaert, D L, et al. “Cardiac Endothelium and Myocardial Function.” Cardiovascular Research, vol. 38, no. 2, 1998, pp. 281-90.
30. Miikkulainen, V, et al. “Crystallinity of Inorganic Films Grown by Atomic Layer Deposition: Overview and General Trends.” Journal of Applied Physics, vol. 113, no. 2, 2013, pp.
31. Rolfe, Barbara, et al. “The Fibrotic Response to Implanted Biomaterials: Implications for Tissue Engineering.” Regenerative Medicine and Tissue Engineering—Cells and Biomaterials, 2011, doi:10.5772/21790.
32. Chen, et al. “Biocompatible Polymer Materials: Role of Protein-Surface Interactions.” Progress in Polymer Science, vol. 33, no. 11, 2008, pp. 1059-1087.
33. Sumpio, et al. “Cells in Focus: Endothelial Cell.” International Journal of Biochemistry and Cell Biology, vol. 34, no. 12, 2002, pp. 1508-1512.
34. Luka, Grzegorz, et al. “Kinetics of Anatase Phase Formation in TiO2 Films during Atomic Layer Deposition and Post-Deposition Annealing.” CrystEngComm, vol. 15, no. 46, 2013, pp. 9949-9954.
35. Anderson, Jm. “Biological Responses to Materials.” Annual Review Of Materials Research, vol. 31, 2001, pp. 81-110.
36. Raimondo, Theresa, et al. “Greater Osteoblast and Endothelial Cell Adhesion on Nanostructured Polyethylene and Titanium.” International Journal of Nanomedicine, vol. 5, no. 1, 2010, pp. 647-652.
37. Seitz, J.-M., et al. “Magnesium Degradation Products: Effects on Tissue and Human Metabolism.” Journal of Biomedical Materials Research Part A, vol. 102, no. 10, 2014, pp. 3744-3753.
38. Li, Jian-Jun, et al. “Impact of C-Reactive Protein on in-Stent Restenosis: a Meta-Analysis.” Texas Heart Institute Journal, vol. 37, no. 1, 2010, pp. 49-57

1. An implantable medical device coated at least in part with a titanium dioxide coating, wherein the coating comprises two or more single atomic layers of titanium dioxide.

2. The implantable medical device of claim 1, wherein the titanium dioxide coating comprises amorphous titanium dioxide.

3. The implantable medical device of claim 1, wherein each of said single atomic layers has a thickness of about 0.4 angstroms.

4. The implantable medical device of claim 1, wherein the coating comprises about 600 to about 3250 single atomic layers of titanium dioxide.

5. The implantable medical device of claim 1, wherein the thickness of the titanium dioxide coating is in the range from about 70 nm to about 130 nm.

6. The implantable medical device of claim 5, wherein the coating comprises about 2500 single atomic layers of titanium dioxide and has a thickness of about 100 nm.

7. The implantable medical device of claim 1, wherein the titanium dioxide coating has an rms surface roughness from about 25 nm to about 65 nm, or from about 30 nm to about 45 nm.

8. The implantable medical device of claim 1, wherein the device comprises a metal or metal alloy coated at least in part with said titanium dioxide coating.

9. The implantable medical device of claim 8, wherein the metal or metal alloy is selected from the group consisting of Mg—Zn, Ti—V—Al, Ti, and Mg.

10. The implantable medical device of claim 1, wherein the device comprises a bioresorbable material coated at least in part with said titanium dioxide coating.

11. The implantable medical device of claim 10, wherein the device is a bioresorbable vascular scaffold.

12. The implantable medical device of claim 1, wherein the implantable medical device is selected from the group consisting of a stent, stimulator, catheter, pacemaker, defibrillator, lead, electrode, bone fixation device, screw, pin, orthopedic implant, dental implant, pump, or prosthesis.

13. The implantable medical device of claim 12, wherein the device is a vascular stent, and wherein the titanium dioxide coating is operative to extend the restoration time and/or the resorption time resulting from the stent when implanted in a vessel.

14. The implantable vascular device of claim 13, wherein the extension of the restoration time and/or the resorption time is modulated by the thickness of the titanium dioxide coating.

15. The implantable medical device of claim 1, wherein the titanium dioxide coating promotes adhesion of mammalian cells to the titanium dioxide coating.

16. The implantable medical device of claim 1, wherein the titanium dioxide coating promotes proliferation of mammalian cells on the titanium dioxide coating.

17. The implantable medical device of claim 1, wherein the titanium dioxide coating inhibits growth of bacteria on the titanium dioxide coating.

18. The implantable medical device of claim 1, wherein the titanium dioxide coating is deposited using two or more cycles of atomic layer deposition (ALD).

19. A method of treating a medical condition in a subject, the method comprising implanting the implantable medical device of claim 1 into the subject's body.

20. The method of claim 19, wherein the medical condition is selected from the group consisting of coronary artery disease, cardiac arrhythmia, a spinal condition, broken bone, torn ligament, a dental condition, urinary obstruction, a prostate condition, cancer, diabetes, and chronic pain.

21. The method of claim 19, wherein adhesion of cells of the subject to the implanted medical device is enhanced by the titanium dioxide coating.

22. The method of claim 19, wherein proliferation of cells of the subject on or near the implanted medical device is enhanced by the titanium dioxide coating.

23. The method of claim 19, wherein growth of bacteria on or near the implanted medical device is enhanced by the titanium dioxide coating.

24. The method of claim 19, wherein healing of a surgical wound is promoted by the titanium dioxide coating or the probability of post-surgical infection is reduced by the titanium dioxide coating.

25. The method of claim 19, wherein the method comprises performing percutaneous coronary intervention (PCI).

26. The method of claim 25, wherein the implantable medical device is a bioresorbable vascular scaffold, and wherein restoration time following PCI is extended by the titanium dioxide coating.

27. The method of claim 19, wherein the method comprises performing orthopedic surgery or a dental procedure.

28. A method of coating a surface of an implantable medical device with a titanium dioxide coating, the method comprising:
(a) providing a medical device comprising a surface to be coated;
(b) performing one cycle of atomic layer deposition to coat at least a portion of the surface with a first atomic layer of titanium dioxide; and
(c) performing one or more additional cycles of atomic layer deposition to coat the first atomic layer of titanium dioxide one or more additional atomic layers of titanium dioxide.

29. The method of claim 28, wherein each atomic layer of titanium dioxide has a thickness of about 0.4 angstrom.

30. The method of claim 28, wherein the coating comprises amorphous titanium dioxide.

31. The method of claim 28, wherein the atomic layer deposition is carried out at a temperature in the range from about 130° C. to about 165° C., or from about 145° C. to about 155° C.

32. The method of claim 28, wherein each cycle of atomic layer deposition comprises:
(i) exposing a surface to be coated to tetrakis(dimethylamido)titanium (TDMATi) gas in a reaction chamber;
(ii) purging the chamber with an inert gas;
(iii) exposing the coating to H₂O; and
(iv) purging the chamber again with an inert gas.

33. The method of claim 32, wherein the exposure to tetrakis(dimethylamido)titanium is performed for about 100 milliseconds.

34. The method of claim 32, wherein the exposure to H₂O is performed for about 100 milliseconds.

35. The method of claim 28, wherein the surface to be coated comprises a metal or metal alloy.

36. The method of claim 35, wherein the metal or metal alloy is selected from the group consisting of Mg—Zn, Ti—V—Al, Ti, and Mg.

37. The method of claim 28, wherein a total of about 600 to about 3250 cycles of atomic layer deposition are performed.

38. The method of claim 37, wherein the total thickness of the titanium dioxide coating is from about 24 nm to about 130 nm.

39. A kit for implanting a coated medical device, the kit comprising the implantable medical device of claim 1 and instructions for use of the device.

40. The kit of claim 39 comprising a plurality of said implantable medical devices, the plurality of devices having a range of different sizes.

41. The kit of claim 39, wherein contents of the kit are packaged and sterile.

42. The kit of claim 39, wherein the kit comprises one or more bioresorbable vascular scaffolds for percutaneous coronary intervention, instructions for use, and optionally one or more further devices for use in performing said percutaneous coronary intervention.
